(19) United States
(12) Patent Application Publication
(10) Pub. No.: US 2026/0295594 A1
(43) Pub. Date: Oct. 1, 2026

(54) CONFIGURABLE ASSEMBLY FOR RETAINING AND DISPENSING LIQUIDS

(71) Applicant: Evolution Design & Development LLC, Perkasie, PA (US)

(72) Inventors: Anthony P. Pagliaro, JR., Perkasie, PA (US); Clemens E. Zoellner, Bay City, MI (US); Scott R. Johnson, Troy, NY (US)

(21) Appl. No.: 19/702,232

(22) Filed: Jun. 9, 2026

Related U.S. Application Data
(62) Division of application No. 18/186,881, filed on Mar. 20, 2023.
(60) Provisional application No. 63/323,150, filed on Mar. 24, 2022.

(51) Int. Cl. B01L 3/00

(52) U.S. Cl. CPC B01L 3/563; B01L 3/567; B01L 2200/023; B01L 2200/026; B01L 2200/0689; B01L 2400/0633

(57) ABSTRACT

A configurable port fitting for a container, such as a bioprocess bag, has one or more O-ring grooves and two opposing locking arms (e.g., 604). The port fitting is mounted onto the container by (i) inserting one end of the port fitting into a port in the container such that O-ring(s) form seals between the O-ring grooves and the container and (ii) rotating the port fitting to engage the locking arms with two opposing retention clips (e.g., 506) of the container. Some port fittings are fixed port fittings that provide a fixed opening into the container. Other port fittings are port sizers that receive a valve connector that enables flow control between closed and open positions. Port fittings of different sizes enable different flow rates. Some port fittings (and other connectors) have a threaded tube barb that receives a threaded tube retainer that secures tubing in place over the barb.

### CROSS-REFERENCE TO RELATED APPLICATIONS

This application claims the benefit of the filing date of U.S. provisional application No. 63/323,150, filed on Mar. 24, 2022 as attorney docket no. 1405.001PROV, the teachings of which are incorporated herein by reference in their entirety.

### BACKGROUND

### Field of the Disclosure

The present disclosure relates to assemblies for retaining and dispensing liquids and, more specifically but not exclusively, to bioprocess bag assemblies, such as (without limitation) those used in medical, laboratory, and cell and gene therapy applications.

### Description of the Related Art

This section introduces aspects that may help facilitate a better understanding of the disclosure. Accordingly, the statements of this section are to be read in this light and are not to be understood as admissions about what is prior art or what is not prior art.

Containers (e.g., flexible bags) for fluid containment have been used in numerous applications including bioprocess applications that require a high degree of assurance that (i) contamination will not enter the closed system and (ii) leakage will not occur.

### BRIEF DESCRIPTION OF THE DRAWINGS

Embodiments of the disclosure will become more fully apparent from the following detailed description, the appended claims, and the accompanying drawings in which like reference numerals identify similar or identical elements.

FIG. 1 is a plan view of a bag assembly 102 according to one embodiment of the present disclosure;

FIG. 2 is a plan view of the bag 104 of FIG. 1 without the bag hangar 106;

FIG. 3 is an enlarged, perspective, X-ray view of the bottom of the bag assembly 102 of FIG. 1;

FIG. 4A is a perspective, bottom-up view of the canoe assembly 402 of FIG. 1;

FIG. 4B is an end view of the canoe assembly 402 of FIG. 4A;

FIG. 4C is a perspective view of the canoe assembly 402 with the three valve connectors 110(1)-110(3) maintained at their “closed” configurations by three tethered safety clips 406;

FIG. 4D is a perspective view of the canoe assembly 402 with the three tethered safety clips 406 disengaged and with the three valve connectors 110(1)-110(3) at their “open” configurations;

FIG. 4E is an exploded, side view of the canoe assembly 402 of FIG. 4C;

FIG. 5 is a perspective view showing the bottom of the canoe 114;

FIG. 6A is a perspective view of the port sizer 108(3);

FIG. 6B is a cross-sectional side view of the port sizer 108(3);

FIG. 6C is a perspective bottom view of the port sizer 108(3);

FIG. 7A is a perspective view of the large valve connector 110(3);

FIG. 7B is a cross-sectional side view of the large valve connector 110(3);

FIGS. 8A-8C show perspective views of small, medium, and large, unthreaded, fixed port fittings 802(1)-802(3), respectively, each of which can be used instead of a corresponding port sizer 108/valve connectors 110 pair;

FIGS. 8D-8F show perspective views of small, medium, and large, threaded, fixed port fittings 808 having threaded tube barbs 810 and stops 612;

FIG. 8G shows a side view of a fixed port fitting 814 having an integral, threaded “wye” tube connector with threaded tube barbs;

FIG. 8H shows a side view of a fixed port fitting 816 having an integral, threaded “tee” tube connector with threaded tube barbs;

FIG. 8I shows a perspective view of a fixed port fitting 802A;

FIG. 9 is a perspective view of a luer lock valve connector 902 having a luer lock 904 instead of a tube barb for mating with a tube (not shown);

FIG. 10 is a side view of block-out plug 1002;

FIGS. 11A-11C respectively show a “tee” valve connector 1102 with two threaded tube barbs 810, a “wye” valve connector 1104 with two threaded tube barbs 810, and a “wye” valve connector 1106 with two valve-connector housings 1108;

FIGS. 12A and 12B are plan views of female and male bag hangar components 1202 and 1204, respectively, which can be used to form the bag hanger 106 of FIG. 1;

FIG. 12C shows a perspective view of the female and male bag hangar components 1202 and 1204 positioned to be configured to the top portion of a bag chamber 112;

FIG. 12D shows a zoomed-in, perspective view of the male bag hangar 1204 configured with the bag chamber 112;

FIG. 12E shows a zoomed-in perspective view of the final bag hangar assembly;

FIG. 13 shows a side view of the bag assembly 102 of FIG. 1 with a bag probe 1302 attached to one of the valve connectors 110;

FIG. 14A is a perspective view of a canoe assembly 402 in its “closed” configuration;

FIG. 14B is a cross-sectional perspective side view of the canoe assembly 402 of FIG. 14A;

FIG. 14C is a cross-sectional perspective end view of the canoe assembly 402 of FIG. 14A;

FIGS. 14D-14F are analogous to the views of FIGS. 14A-14C, respectively, but with the canoe assembly 402 in its “open” configuration with the ends of the valve connectors 110 extending above the top surface of the canoe 114;

FIG. 15A is a cross-sectional end view of the canoe assembly 402 of FIG. 14A in its “closed” configuration, and FIG. 15B is a cross-sectional end view of the canoe assembly 402 of FIG. 14D in its “open” configuration.

FIG. 16A is a zoomed-in, cross-sectional side view of the canoe assembly 402 of FIG. 14A with the port sizer locking clips 606 engaged with the valve connector “closed” locking tabs 706;

FIG. 16C is a zoomed-in, cross-sectional side view of the canoe assembly 402 of FIG. 14D with the port sizer locking clips 606 engaged with the valve connector “open” locking tabs 708;

FIG. 16B is a zoomed-in, cross-sectional side view of the same canoe assembly 402 transitioning from the “closed” configuration of FIG. 14A to the “open” configuration of FIG. 14D;

FIGS. 17A and 17B show perspective views of the canoe 114 and the port sizer 108(3), respectively;

FIGS. 17C and 17D show different, zoomed-in, cutaway, perspective views, and FIG. 17E shows a zoomed-in, perspective view of the canoe 114 and port sizer 108(3) of FIGS. 17A and 17B;

FIGS. 18A-18C show different stages of connecting different sized tubes 1802 to different sized, threaded, fixed port fittings 808 using different sized, threaded tube retainers 1404;

FIG. 19A is a side view of half of a threaded fixed port fitting 808, and FIG. 19B is a cross-sectional side view of the threaded fixed port fitting 808 of FIG. 19A;

FIG. 20A shows a perspective view of a port sizer 108, a valve connector 110, and a tethered safety clip 406 before they are assembled together;

FIG. 20B shows a perspective view of the elements of FIG. 20A after the tether ring 408 has been placed over the port sizer stop 612;

FIG. 20C shows a perspective view of the elements of FIG. 20B after the tether ring 408 has been rotated clockwise about 90 degrees with respect to the port sizer 108;

FIG. 20D shows a perspective view of the elements of FIG. 20C after the tether clip 410 has been rotated 180 degrees with respect to the tether ring 408, resulting in a bending of the tether 412.

FIG. 20E shows a perspective view of the elements of FIG. 20D and a dust cap 414 after (i) the valve connector 110 has been inserting into the port sizer 108 up to the “closed” configuration and (ii) the tether clip 410 has been pushed onto the valve connector 110 just below the port sizer stop 612 and just above the valve connector stop 710;

FIG. 20F shows a perspective view of the elements of FIG. 20E and a tube retainer 414 after (i) the dust cap 414 has been placed over the barbed end of the threaded tube barb 810 (not visible in FIG. 20F) and (ii) a tube retainer 1404 has been screwed onto the threaded tube barb 810;

FIG. 21 shows a perspective view of the bottom of a canoe assembly 402 having three port sizer/valve connector sub-assemblies in three different configurations;

FIG. 22A is a perspective view of a tube retainer 1404, and FIG. 22B is a cross-sectional perspective view of the tube retainer 1404 of FIG. 22A;

FIG. 23 is a zoomed-in cross-sectional perspective view of a tube retainer X046 screwed onto a threaded tube barb 810 with an intervening dust cap 414;

FIG. 24 is a cross-sectional side view of a tube retainer 1404 fully screwed onto a threaded tube barb 810 with a tube 1802 secured and sealed in place;

FIG. 25 is a perspective view of a canoe assembly 2502 having a two-port canoe 2504;

FIG. 26A is an exploded perspective view of a single-fitting port 2602 configured to receive a single port fitting;

FIG. 26B is a top-down view of a two-fitting port 2616 configured to receive two port fittings, and FIG. 26C is a top-down view of a four-fitting port 2618 configured to receive four port fittings;

FIGS. 26D and 26E are side and cross-sectional (through the center line) views, respectively, of the two-fitting port 2616 of FIG. 26B with two fixed port fittings 2604 and a gasket 2614;

FIGS. 27A and 27B are exploded and assembled views, respectively, showing a side view of the fixed port fitting 2604 and a cross-sectional view of a combined gasket/O-ring 2620;

FIG. 27C is a cross-sectional side view of the fixed port fitting 2604;

FIG. 28 is a partially exploded view of the fixed port fitting 2604 and the single-fitting port 2602 mated with a cap 2802 for a bottle or carboy;

FIG. 29A is a side view of a dip tube 2902 having a dip tube fitting 2904 with two O-ring grooves 2906, and FIG. 29B is a side view of a dip tube 2908 having a dip tube fitting 2910 with single O-ring groove 2906;

FIG. 30A is a perspective view of a filling needle 3002, FIG. 30B is a perspective view of the filling needle 3002 with a dust cap 414, and FIG. 30C is a perspective view of the filling needle 3002 with both the dust cap 414 and a threaded tube retainer 1404;

FIGS. 30D-30F are perspective views representing a tube 1802 being mounted onto the filling needle 3002 of FIGS. 30A-30C using the threaded tube retainer 1404;

FIG. 31 is a side view of a vented port fitting 3102;

FIGS. 32A, 32B, 32C, and 32D show top-down, end, X-ray side, and cross-sectional side views of a three-port canoe 3202 according to an alternative embodiment;

FIG. 32E is an exploded side view of the three-port canoe 3202 of FIGS. 32A-32D ready to receive three fixed port fittings 2604;

FIGS. 33A and 33B are side views of two different tube-to-tube connectors;

FIG. 33C is a side view of a two-piece tube-to-tube connector 3306 that provides flow-valve functionality;

FIGS. 33D-33F show side views of retainer-ready cross, tee, and wye tube-to-tube connectors, respectively;

FIGS. 33G and 33H show side views of retainer-ready tri-clamp tube barb connectors;

FIGS. 331 and 33J show side views of two different three-piece tube-to-tube TEE connectors that provide flow-valve functionality at their lower ports;

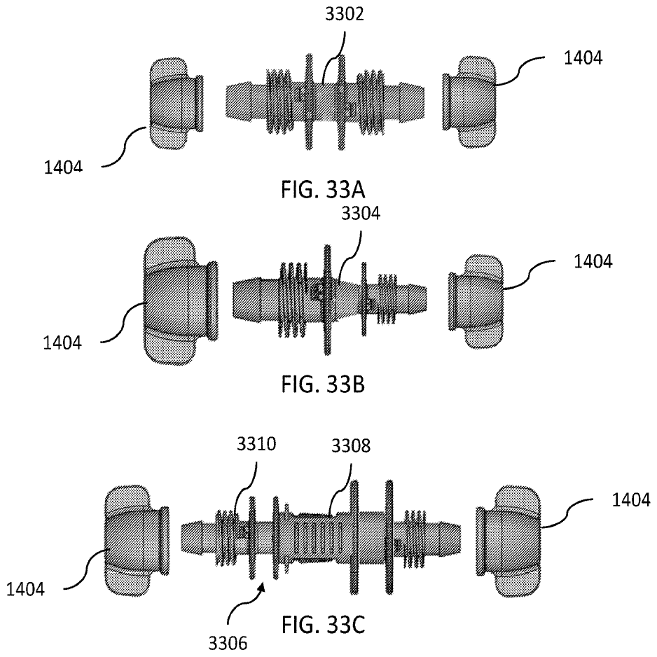

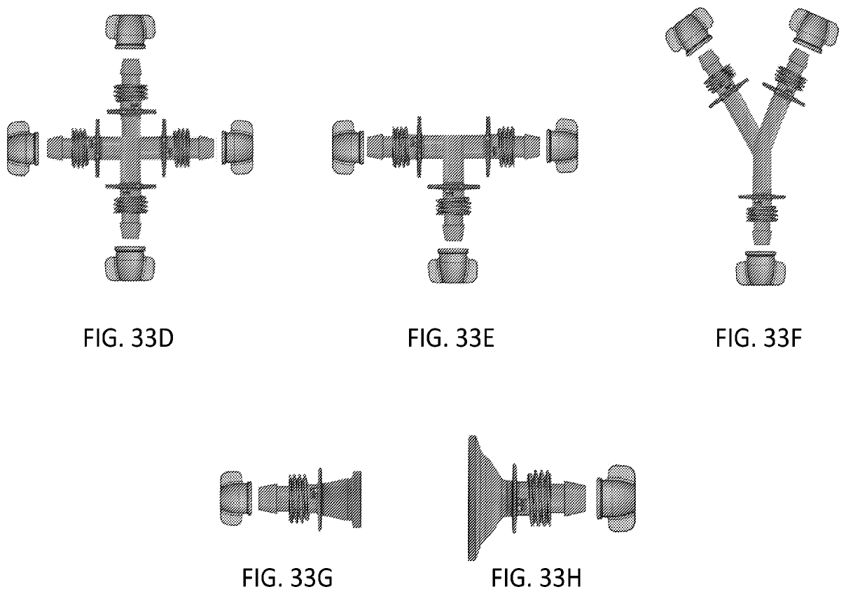

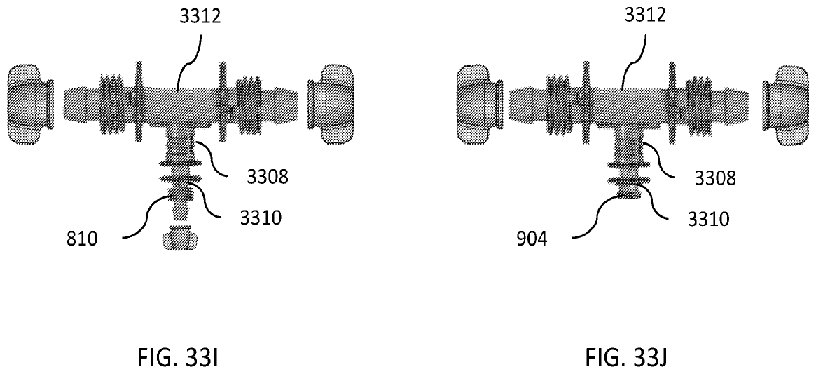

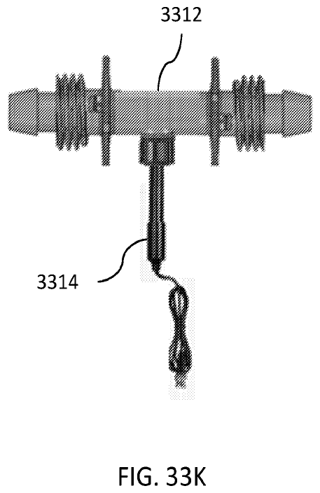

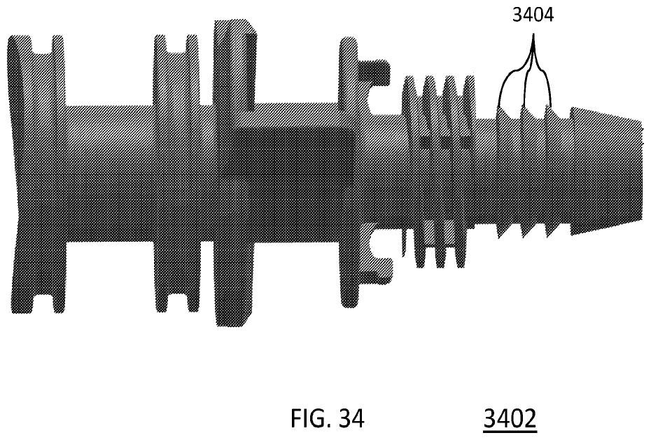

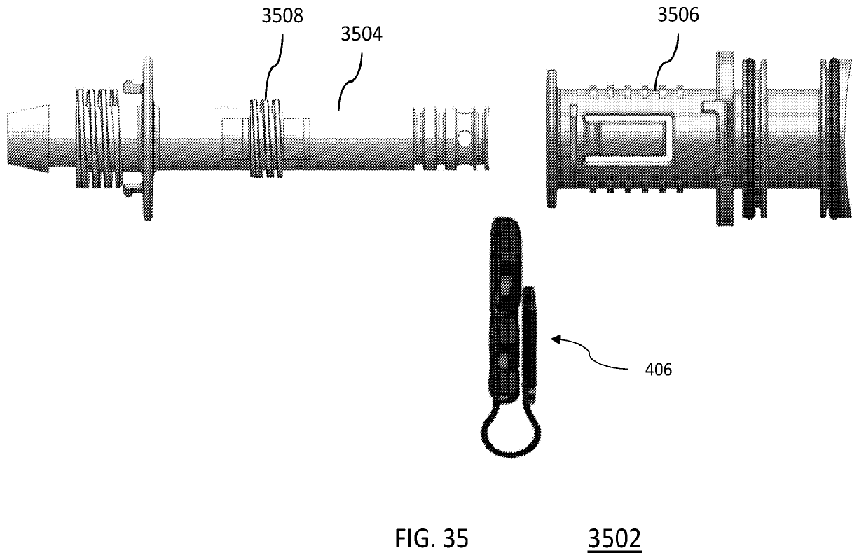

FIG. 33K is a side view of the three-port tube-to-tube connector analog 3312 of FIGS. 331 and 33J with a probe 3314 connected to the lower port to enable fluid path measurements;

FIG. 34 is a side view of a fixed port fitting 3402 having a threaded tube barb with three tube grippers 3404; and

FIG. 35 is an exploded side view of a reconfigurable assembly 3502.

### DETAILED DESCRIPTION

Detailed illustrative embodiments of the present disclosure are disclosed herein. However, specific structural and functional details disclosed herein are merely representative for purposes of describing example embodiments of the present disclosure. The present disclosure may be embodied in many alternate forms and should not be construed as limited to only the embodiments set forth herein. Further, the terminology used herein is for the purpose of describing particular embodiments only and is not intended to be limiting of example embodiments of the disclosure.

As used herein, the singular forms “a,” “an,” and “the,” are intended to include the plural forms as well, unless the context clearly indicates otherwise. It further will be understood that the terms “comprises,” “comprising,” “contains,” “containing,” “includes,” and/or “including,” specify the presence of stated features, steps, or components, but do not preclude the presence or addition of one or more other features, steps, or components. It also should be noted that in some alternative implementations, the functions/acts noted may occur out of the order noted in the figures. For example, two figures shown in succession may in fact be executed substantially concurrently or may sometimes be executed in the reverse order, depending upon the functions/acts involved.

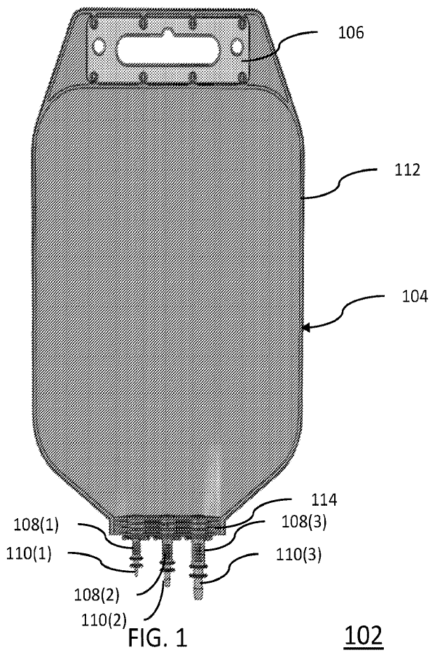

FIG. 1 is a plan view of a bag assembly 102 according to one embodiment of the present disclosure. The bag assembly 102 includes bag 104, bag hangar 106, three port sizers 108(1)-108(3), and three valve connectors 110(1)-110(3), where the bag 104 includes flexible bag chamber 112, bag hangar 106, and three-port canoe 114. Although the bag assembly 102 has a bag 104 of a certain size and a canoe 114 with three ports, those skilled in the art will understand that alternative embodiments of the present disclosure may have bags or different sizes and/or canoes having fewer or more ports.

As used herein, the term “port sizer” refers to a type of port fitting that receives a valve connector that supports both (i) a “closed” configuration in which the valve connector is inserted into the port sizer to a “closed position” at which fluid cannot flow through the port sizer and (ii) an “open” configuration in which the valve connector is inserted into the port sizer to an “open position” at which fluid can flow through the port sizer. Another type of port fitting, referred to herein as a “fixed port fitting,” is permanently in an open configuration and is not designed to receive a valve connector.

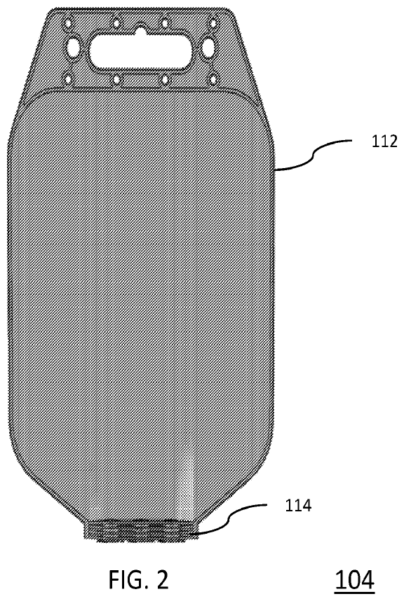

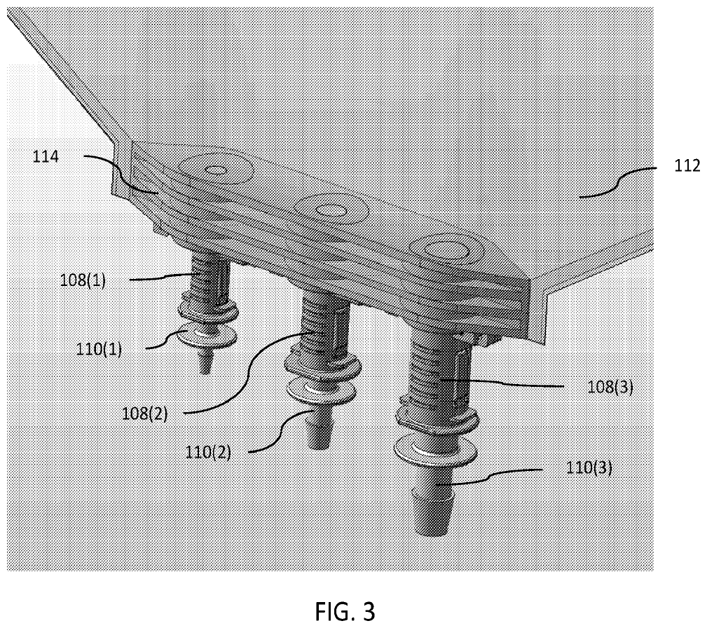

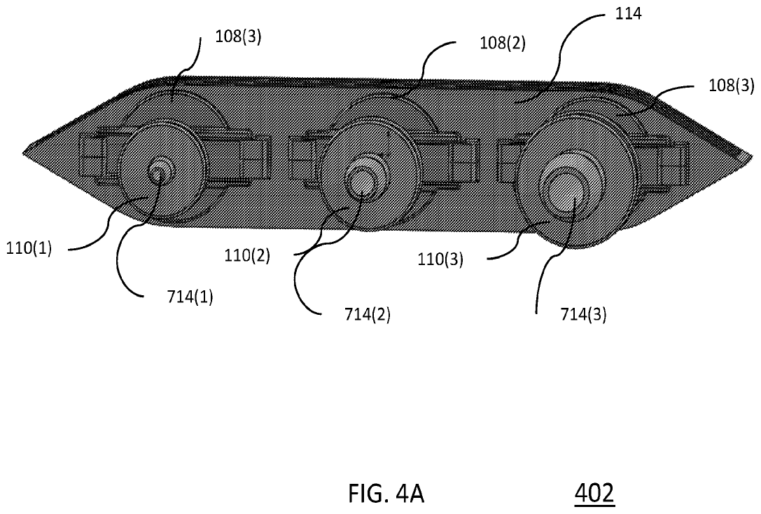

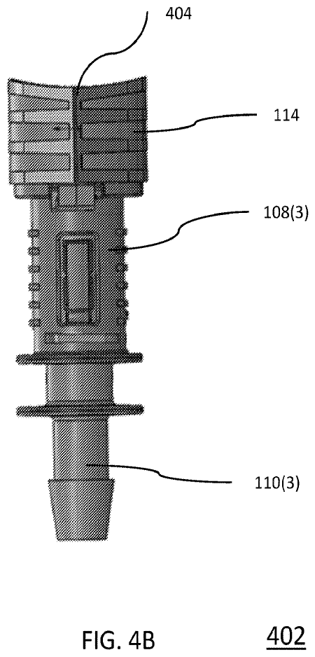

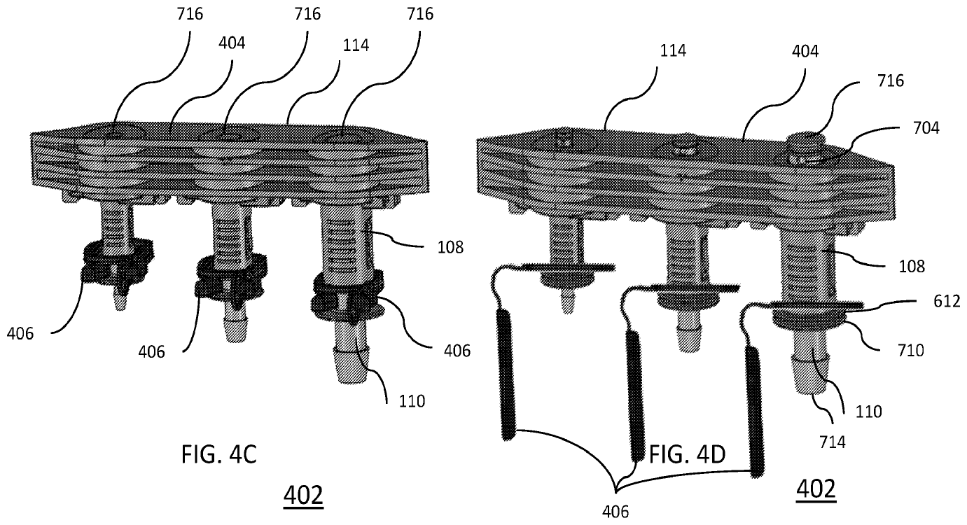

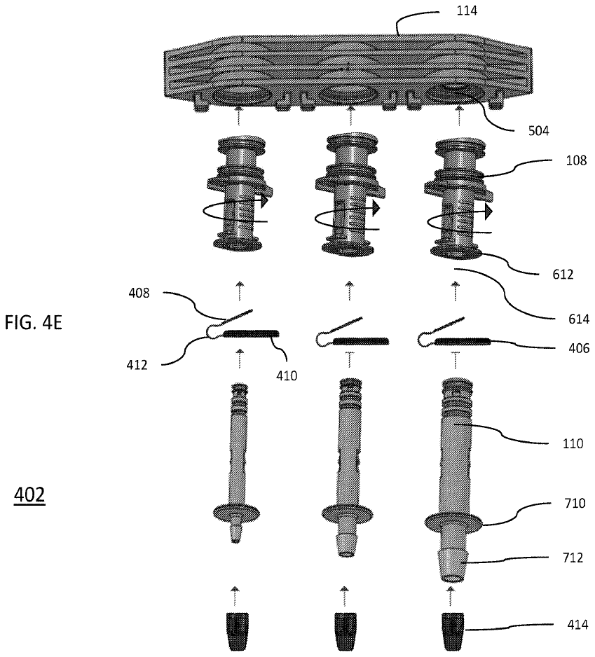

FIG. 2 is a plan view of the bag 104 of FIG. 1 without the bag hangar 106 and without any port sizers/fittings inserted into the canoe 114. FIG. 3 is an enlarged, perspective, X-ray view of the bottom of the bag assembly 102 of FIG. 1 showing the canoe 114, the three port sizers 108(1)-108(3), and the three valve connectors 110(1)-110(3). FIG. 4A is a perspective, bottom-up view of the canoe assembly 402 of FIG. 1 comprising the canoe 114, the three port sizers 108(1)-108(3), and the three valve connectors 110(1)-110(3). FIG. 4B is an end view of the canoe assembly 402 of FIG. 4A showing the port sizer 108(3) locked into the canoe 114, and the valve connector 110(3) inserted into, but not fully engaged with, the port sizer 108(3).

As known in the art, two pieces of a single-layer or multi-layer flexible plastic, fluoropolymer, or silicone film or some combination are assembled, e.g., by welding, to form the bag chamber 112. A single-port or multi-port, rigid, plastic or fluoropolymer, injection-molded canoe, such as the three-port canoe 114, is then assembled into an unwelded end of the bag chamber 112, e.g., by welding, to form a bag, such as the bag 104. Canoes of the present disclosure may be made of other suitable materials, such as (without limitation) thermoplastic elastomers (TPEs), thermoplastic polyurethane (TPU), or fluoropolymers, and/or manufactured using other suitable techniques, such as (without limitation) 3D printing.

The particular bag assembly 102 of FIG. 1 is configured with three differently sized pairs of port sizers 108 and valve connectors 110 sized to receive tubes (not shown) of three different sizes (e.g., having three different inner diameters). In particular, the bag assembly 102 has:
Small port sizer 108(1) and small valve connector 110(1) sized to receive a tube having, e.g., a ⅛-inch inner diameter;
Medium port sizer 108(2) and medium valve connector 110(2) sized to receive a tube having, e.g., a ¼-inch inner diameter; and
Large port sizer 108(3) and large valve connector 110(3) sized to receive a tube having, e.g., a ⅜-inch inner diameter.
   This provides the user the opportunity to select and customize the internal diameter of the fluid flow and thus the tube size. Those skilled in the art will understand that these sizes are examples and that other suitable sizes are possible.

As shown in FIG. 4A and as described in further detail below, the small, medium, and large valve connectors 110(1)-110(3) have small, medium, and large channels 714(1)-714(3), respectively, that enable different rates of fluid to flow between the interior of the bag 104 and the tubes in either direction. In other bag assemblies of the present disclosure, the bag 104 may be configured with any combination of three differently sized or same sized pairs of port sizers 108 and valve connectors 110.

As shown in FIG. 4B, the top surface 404 of the canoe 114 has a convex, groove-shaped curvature that provides a relatively smooth transition between the inner wall of the bag chamber 112 and the canoe top surface 404 that facilitates the removal of most if not all of the fluid in the bag 104 and/or eliminating fluid retain. Bioprocess fluids are typically expensive and any fluid that undesirably remains in the bag 104 may have significant value.

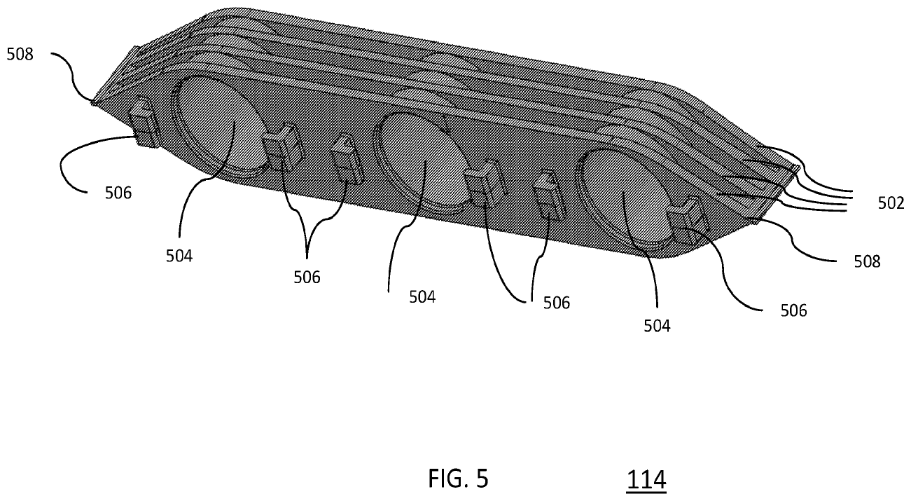

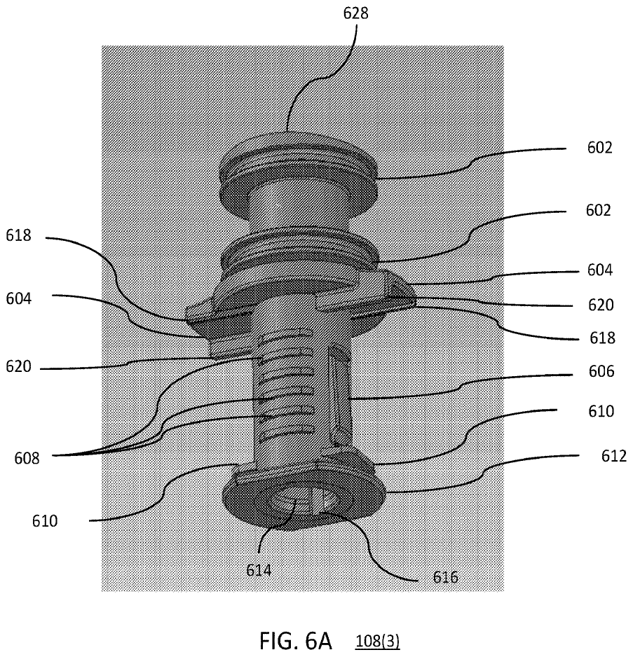

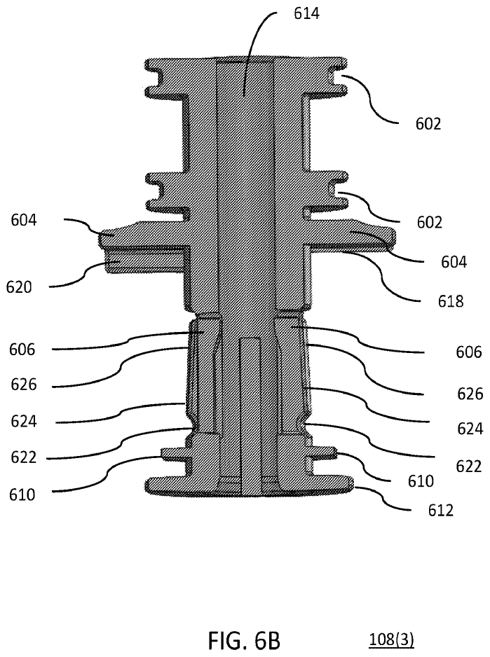

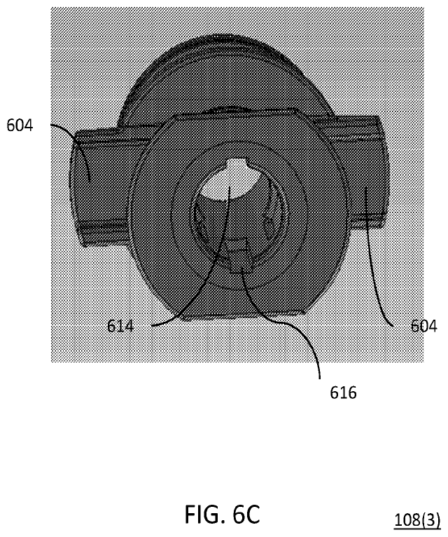

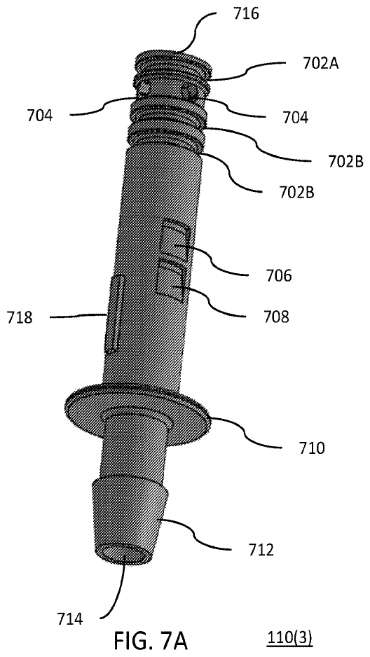

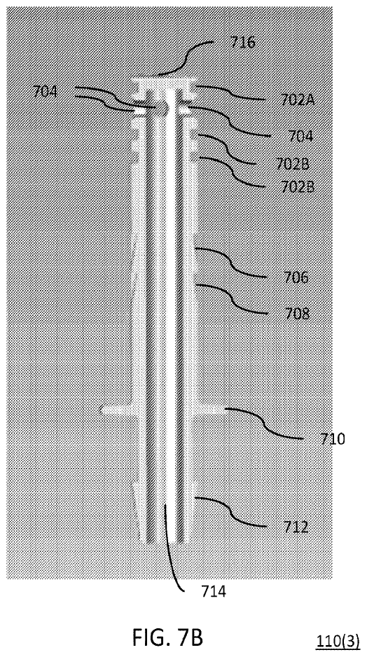

FIG. 4C is a perspective view of the canoe assembly 402 with the three valve connectors 110(1)-110(3) maintained at their “closed” configurations by three tethered safety clips 406. FIG. 4D is a perspective view of the canoe assembly 402 with the three tethered safety clips 406 disengaged and with the three valve connectors 110(1)-110(3) at their “open” configurations. FIG. 4E is an exploded, side view of the canoe assembly 402 of FIG. 4C. FIG. 5 is a perspective view showing the bottom of the canoe 114. FIG. 6A is a perspective view of the large port sizer 108(3). FIG. 6B is a cross-sectional side view of the large port sizer 108(3). FIG. 6C is a perspective bottom view of the large port sizer 108(3). FIG. 7A is a perspective view of the large valve connector 110(3). FIG. 7B is a cross-sectional side view of the large valve connector 110(3).

As shown in FIG. 5, the canoe 114 has (e.g., four) weld bars 502 that provide surfaces for welding the canoe 114 to the bag chamber 112 (FIG. 1) and three equally sized cylindrical through-holes (i.e., ports) 504, each of which has a corresponding pair of retention clips 506. Canoe 114 has tapered ends 508, which provide a sloping angle transition for welding the film of the bag chamber 112 against the canoe 114, thereby reducing the chance leak points to be created.

As shown in FIGS. 6A-6C for the large port sizer 108(3), each (e.g., rigid thermoplastic or fluoropolymer molded) port sizer 108 has two O-ring grooves 602 for receiving two respective (e.g., silicone or thermoplastic elastomer) O rings (not shown) to prevent leakage, two opposing locking arms 604, two opposing locking clips 606, a number of anti-slip grips 608, two tether tabs 610, a stop 612, and a cylindrical channel 614 running through the port sizer 108 from top to bottom. As explained further below, the bottom end of the port sizer channel 614 has a keyway 616. Note that the upper portions of the three differently sized port sizers 108(1)-108(3) of FIG. 1 have the same outer diameters (to enable any size port sizer 108 to be mounted into any of the three equally sized canoe through-holes 504), while the outer diameters of their lower portions are differently sized.

Each locking arm 604 has a detent 618 and a rotation stop 620. As shown in FIG. 6A and as described further below, the detents 618 and rotation stops 620 are configured on opposing sides of the locking arms 604 to enable the locking arms to engage the two corresponding canoe retention clips 506 when the port sizer 108 is permanently mounted into a canoe through-hole 504.

Port sizer 108(3) has two opposing tether tabs 610, which, as described further below, assist in retaining the tethered safety clip 406.

As shown most clearly in FIG. 6B, each port sizer locking clip 606 is a cantilevered structure that is connected to the rest of the port sizer 108 at only the bottom of the locking clip 606 at a relatively thin connection 622 that enables the cantilevered structure to rotate relative to that connection point. As described further below, the lower portion 624 of the locking clip 606 has a vertical inner surface, while the upper portion 626 has a beveled inner surface.

As shown in FIGS. 7A and 7B for the large valve connector 110(3), each (e.g., rigid plastic or fluoropolymer molded or 3D printed) valve connector 110 has one upper O-ring groove 702A and two lower O-ring grooves 702B for receiving three respective (e.g., silicone or thermoplastic elastomer) O rings (not shown) to prevent leakage, one or more (in this embodiment, four) side ports 704, two opposing, “closed” locking tabs 706 (only one of which is visible in FIG. 7A), two opposing, “open” locking tabs 708 (only one of which is visible in FIG. 7A), a stop 710, a tube barb 712, and a channel 714 running through the valve connector 110 from the side ports 704 at the top to the open bottom that allows fluid to flow into or out of the bag chamber 112 through the valve connector 110. Note that the top surface 716 of the valve connector 110 has a convex shape that matches the convex shape of the canoe top surface 404 (FIG. 4B) and the convex shape of the top 628 of the port sizer (as shown in FIG. 6A) to form a smooth curvilinear surface when the port sizer is mounted onto the canoe and the valve connector is inserted into the port sizer up to the closed position.

Note that the numbers of O-ring grooves in the port sizers 108 and/or in the valve connectors 110 may be different in different embodiments of the present disclosure from those shown in the figures.

As shown in FIGS. 4C and 4E and as described further below, each (e.g., plastic, TPU, TPE, EPDM, rubber, silicone, elastomeric material, or fluoropolymer molded) tethered safety clip 406 has a (e.g., semi-rigid) tether ring 408 and a (e.g., relatively rigid) tether clip 410 interconnected by a (e.g., flexible) tether 412. Since these elements of the tethered safety clip 406 are formed of the same material, the relative rigidity/flexibility of these elements is a function of their relative thicknesses.

As shown in the “closed” configuration of FIG. 4C, the convex top surface 716 of each valve connector 110 is flush with the convex canoe top surface 404 with the corresponding tethered safety clip 406 preventing the valve connector 110 from transitioning from the “closed” configuration of FIG. 4C to the “open” configuration of FIG. 4D.

As shown in the “open configuration of FIG. 4D, after removing each tethered safety clip 406, the corresponding valve connector 110 can be pushed further into the port sizer 108 until the valve connector stop 710 abuts the port sizer stop 612, at which position, the valve connector top surface 716 extends above the canoe top surface 404, thereby exposing the valve connector side ports 704 and enabling fluid to flow between the interior of the bag 104 and the valve connector channel 714.

As represented in FIG. 4E and as described further below, the canoe assembly 402 of FIG. 4C may be assembled by:
Placing a dust cap 414 (not shown in FIG. 4C) over the barbed end of the tube barb 712 of each valve connector 110;
Looping the tether ring 408 of each tethered safety clip 406 around and over the port sizer stop 612 at the bottom of the corresponding port sizer 108;
Inserting each valve connector 110 (configured with three O-rings) into the channel 614 of the corresponding port sizer 108;
Attaching the tether clip 410 of each tethered safety clip 406 to the corresponding valve connector 110 just above the valve connector stop 710 and just below the port sizer stop 612; and
Mounting each port sizer 108 (configured with two O-rings) into the corresponding canoe through-hole 504.

Note that the canoe assembly 402 of FIG. 4C can be alternatively assembled by performing these steps in different appropriate sequences.

To insert a valve connector 110 into a correspondingly sized port sizer 108, the top portion of the valve connector 110 (configured with three O-rings) is inserted into the bottom end of the port sizer 108 with the valve connector keyway guide 718 aligned to engage the port sizer keyway 616, which in turn aligns the valve connector locking tabs 706 and 708 with the port sizer locking clips 606.

When the valve connector 110 is initially inserted into the port sizer 108 to the “closed” configuration, the port sizer locking clips 606 will engage with the valve connector “closed” locking tabs 706. In particular, as the valve connector 110 proceeds along the port sizer channel 614, the port sizer locking clips 606 will pivot outwardly about their locking clip connections 622 as the outer surface of valve connector 110 pushes outwardly on the beveled surfaces of the locking clips 606 until the “closed” configuration is reached, at which point, the beveled upper portions of the locking clips 606 will be received within the beveled valve connector “closed” locking tabs 706, which prevents subsequent removal of the valve connector 110 from the port sizer 108. In this “closed” configuration, the valve connector's uppermost O-ring is still within the port sizer channel 614 such that fluid flow into and out of the bag 104 through the valve connector 110 is prevented. In addition, in this “closed” configuration, the convex valve connector top surface 716 is flush with the convex canoe top surface 404 (FIG. 4B) to form a contiguous convex groove at the bottom of the bag 104. Through the use of a rotational thread pattern external to the valve connector (110) and a corresponding threaded groove on the internal diameter of the port sizer (108), the valve can be rotated up into an open position, allowing fluid flow. The same feature on the valve connector would then also allow the valve connector to be retracted into a closed position.

Note that the port sizer locking clips 606 and the valve connector “closed” locking tabs 706 are designed to prevent the valve connector 110 from being removed from the port sizer 108 after the “closed” configuration has been achieved. In alternative embodiments, the valve connectors and the port sizers may be designed such that the valve connectors are removable from the port sizers.

As described further below, after achieving the “closed” configuration and with the tether clip 410 of the tethered safety clip 406 disengaged from the valve connector 110, the “open” configuration can be achieved by pushing the valve connector 110 further into the port sizer 108 such that the port sizer locking clips 606 will transition from engaging with the valve connector “closed” locking tabs 706 to be engaged with the valve connector “open” locking tabs 708 in a manner analogous to the initial transition to the “closed” configuration. At this “open” configuration, the valve connector stop 710 abuts the port sizer stop 612, thereby preventing the valve connector 110 from being pushed any further into the port sizer 108 in order to prevent leakage.

Note that the port sizer locking clips 606 and the valve connector “open” locking tabs 708 are designed to prevent the valve connector 110 from being removed from the “open” configuration after the “open” configuration has been achieved. In alternative embodiments, the valve connectors and the port sizers may be designed (i) to enable the valve connectors to transition from the “open” configuration back to the “closed” configuration and (ii) possibly to enable the valve connectors to be removable from the port sizers.

To mount a port sizer 108 into a canoe through-hole 504, the top portion of the port sizer 108 (configured with two O-rings) is inserted into the canoe through-hole 504 until the port sizer locking arms 604 abut the bottom surface of the canoe 114 and then the inserted port sizer 108 is rotated clockwise to engage the two port sizer locking arms 604 with the two corresponding canoe retention clips 506 to keep the port sizer 108 in place within the canoe through-hole 504. Note that the port sizer locking arms 604 have rotation stops 620 that stop the clockwise rotation of the port sizer 108 with respect to the canoe 114 to retain the top curvature profile when the rotation stops 620 abut the sides of the canoe retention clips 506. Note further that the locking arms 604 has detents 618 that prevent the mounted port sizer 108 from being removed from the canoe 114. The main functions of the port sizers 108 are to determine the inside diameter of the fluid path and, in some instances, to house the function of the valve connectors 110. The valve connectors allow the operator to open the bag 104 for fluid input and removal.

Referring again to the bag assembly 102 of FIG. 1, as known in the art, up to three tubes (not shown) of different size can be connected and secured to the three valve connectors 110, for example, using tie wraps, Barblocks, Oetiker clamps, hose clamps, wires, or any other suitable tubing retaining device (not shown) wrapped around the tubes above the barbed ends of the tube barbs 712. The bag assembly 102 of FIG. 1 can then be used to move fluids into or out of the bag 104 at any combination of flow rates supported by the differently sized port sizer/valve connector/tube sub-assemblies by disengaging the tether clip 410 of each corresponding tethered safety clip 406 (if present) and moving each corresponding valve connector 110 from its “closed” configurations to its “open” configuration.

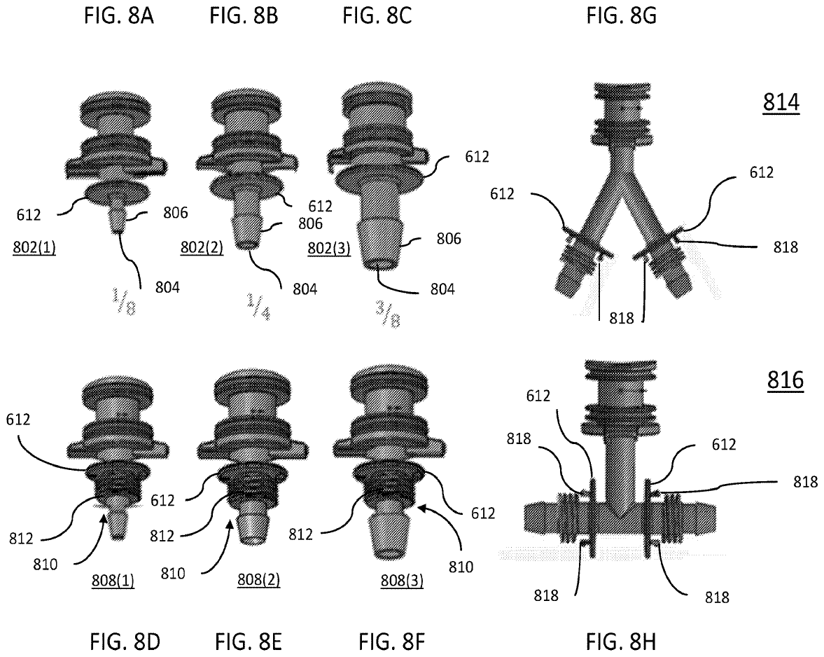

FIGS. 8A-8C show perspective views of small, medium, and large, unthreaded, fixed port fittings 802(1)-802(3), respectively, each of which can be used instead of a corresponding port sizer 108/valve connectors 110 pair. Each fixed port fitting 802 is a unitary structure having a single, fixed “open” configuration and a channel 804 that runs entirely through the fixed port fitting 802 from top to bottom. Each fixed port fitting 802 has (i) two O-ring grooves and two opposing locking arms that are analogous to the corresponding elements of the corresponding port sizer 108 and (ii) a stop 612 and a tube barb 806 that are analogous to the corresponding elements of the corresponding valve connector 110. Since they are always in their “open” configuration, fixed port fittings 802 can be used with conventional tube clamps to control the flow of fluid into and out of the bag 104.

FIGS. 8D-8F show perspective views of small, medium, and large, threaded, fixed port fittings 808 having threaded tube barbs 810 with threading 812. Otherwise, the threaded, fixed port fittings 808 are similar to the corresponding “unthreaded,” fixed port fittings 802 of FIGS. 8A-8C.

FIG. 8G shows a side view of a fixed port fitting 814 having an integral, threaded “wye” tube connector with threaded tube barbs, and FIG. 8H shows a side view of a fixed port fitting 816 having an integral, threaded “tee” tube connector with threaded tube barbs. As shown in FIGS. 8G and 8H and as described further below, each port fitting stop 612 has two opposing retainer locking tabs 818.

FIG. 8I shows a perspective view of a fixed port fitting 802A according to an alternative embodiment. As shown in FIG. 8I, port fitting 802A has extended locking arms 803 (only one of which is seen in FIG. 8I) that extend to the stop 612 that make it easier to rotate the port fitting 802A into a canoe 114, thereby improving the operator's ability to engage the locking mechanism of the port fitting 802A. Analogous extended locking arms can be incorporated into other port fittings of the present disclosure.

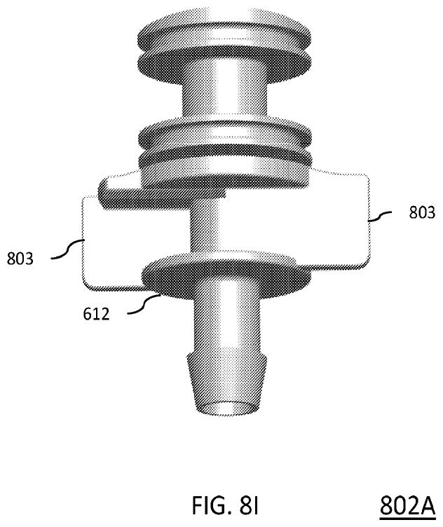

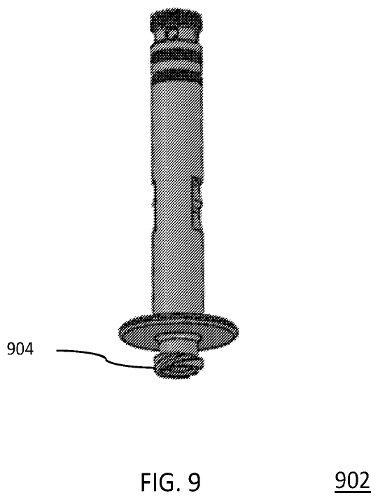

FIG. 9 is a perspective view of a luer lock valve connector 902 having a luer lock 904 instead of a tube barb for mating with a tube (not shown).

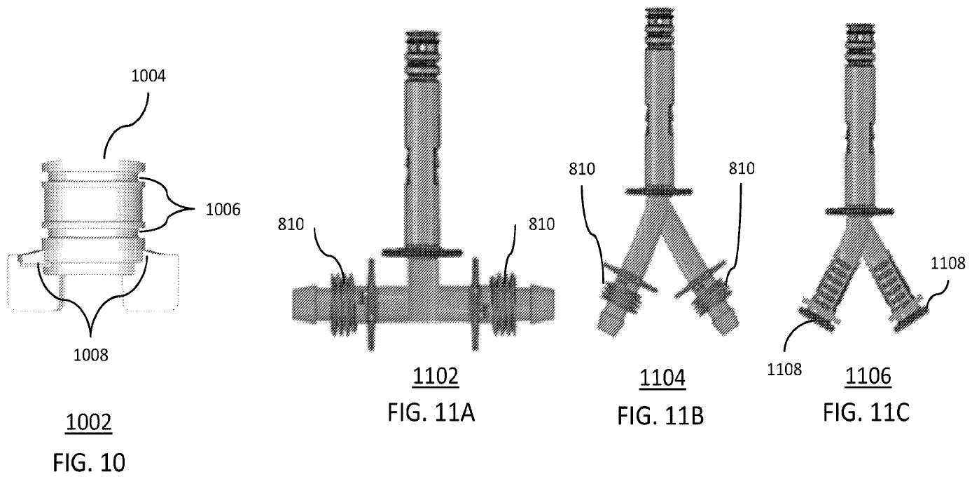

FIG. 10 is a block-out plug 1002 that can be used instead of a port sizer 108/valve connector 110 combination or a fixed port fitting 802/808 to permanently close a canoe through-hole 504 to effectively reduce the number of operable ports in the canoe, if needed. The block-out plug 1002 has a curved top surface 1004, O-ring grooves 1006, and locking arms 1008 analogous to the locking arms 604 of the port fittings 108, 802, and 808.

FIGS. 11A-11C respectively show a “tee” valve connector 1102 having two threaded tube barbs 810, a “wye” valve connector 1104 having two threaded tube barbs 810, and a “wye” valve connector 1106 having two valve-connector housings 1108, where each valve-connector housing 1108 can receive a valve connector (not shown). Each of these three connectors 1102-1106 can be configured with a corresponding port sizer 108 in either a “closed” configuration or an “open” configuration analogous to those described previously.

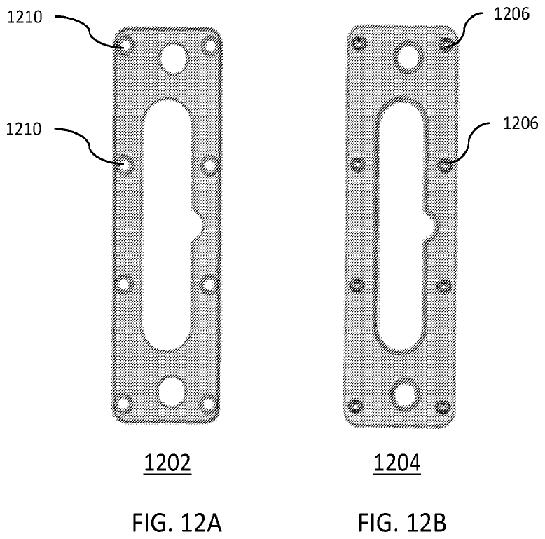

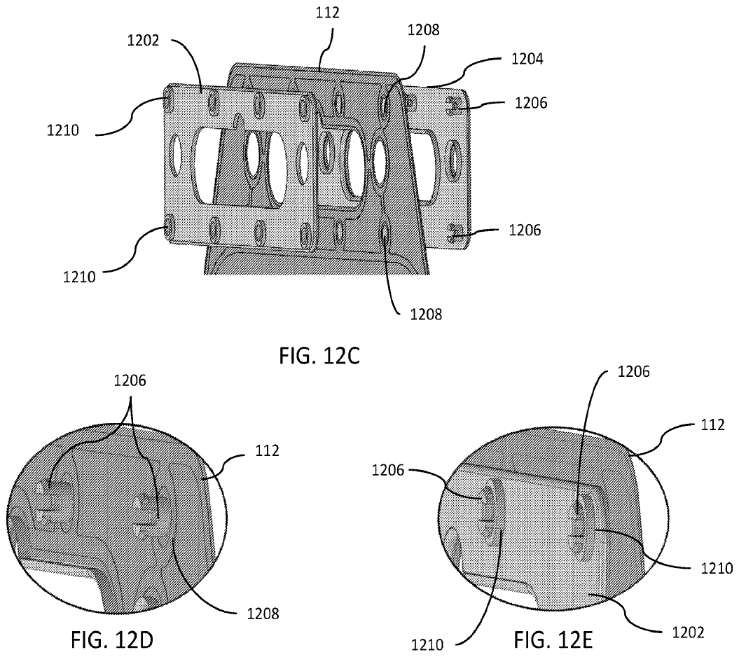

FIGS. 12A and 12B are plan views of female and male bag hangar components 1202 and 1204, respectively, which can be used to form the bag hanger 106 of FIG. 1. FIG. 12C shows a perspective view of the female and male bag hangar components 1202 and 1204 positioned to be configured to the top portion of a bag chamber 112, where the top portion is outside of the bag enclosure that holds fluid. FIG. 12D shows a zoomed-in, perspective view of the male bag hangar component 1204 configured with the bag chamber 112, while FIG. 12E shows a zoomed-in perspective view of the final bag hangar assembly. As shown in FIGS. 12A-12E, the male bag hanger component 1204 has barbed male elements 1206 that are pushed through corresponding openings 1208 in the bag chamber 112 and then through corresponding female elements 1210 in the female bag hangar component 1202 to form and secure the bag hangar 106 (FIG. 1) onto the bag chamber 112. Those skilled in the art will understand that the bag hangar 106 is used to reinforce larger bags 104 from the full weight of the liquid contents.

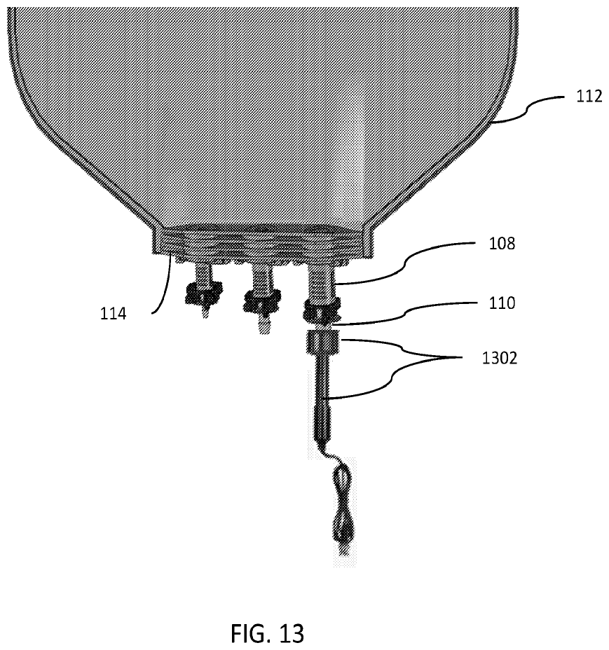

FIG. 13 shows a side view of the bag assembly 102 of FIG. 1 with a bag probe 1302 attached to one of the valve connectors 110. Depending on the configuration, the bag probe 1302 can be used to measure any suitable characteristic(s) of fluid retained in the bag, such as (without limitation) temperature, pH, salinity, dissolved oxygen level, viable cell density, culture viability, growth rate, and/or nutrient levels. Another version includes the probe assembled directly into the port sizer without the need to move the probe in or out of the bag.

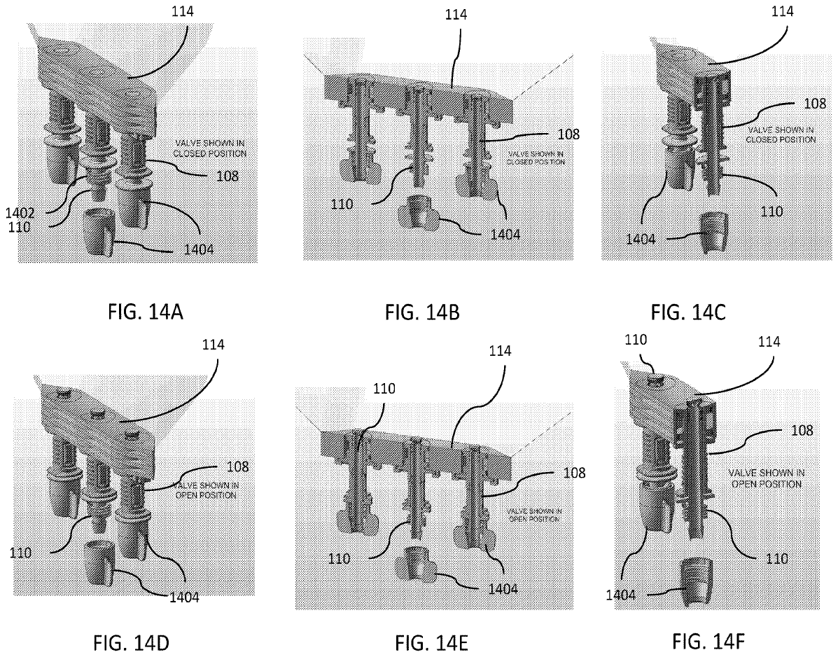

FIG. 14A is a perspective view of a canoe assembly 402 in its “closed” configuration where (i) the valve connectors 110 have threaded tube barbs 810 with threading 1402 and (ii) threaded tube retainers 1404 engage with the threaded tube barbs 810 to assist in sealing and retaining a tube (not shown) onto the barbed end of the threaded tube barb 810. FIG. 14B is a cross-sectional perspective side view of the canoe assembly 402 of FIG. 14A, and FIG. 14C is a cross-sectional perspective end view of the canoe assembly 402 of FIG. 14A.

FIGS. 14D-14F are analogous to the views of FIGS. 14A-14C, but with the canoe assembly 402 in its “open” configuration with the ends of the valve connectors 110 extending above the top surface of the canoe 114. Note that the tubes are not shown in FIGS. 14A-14F.

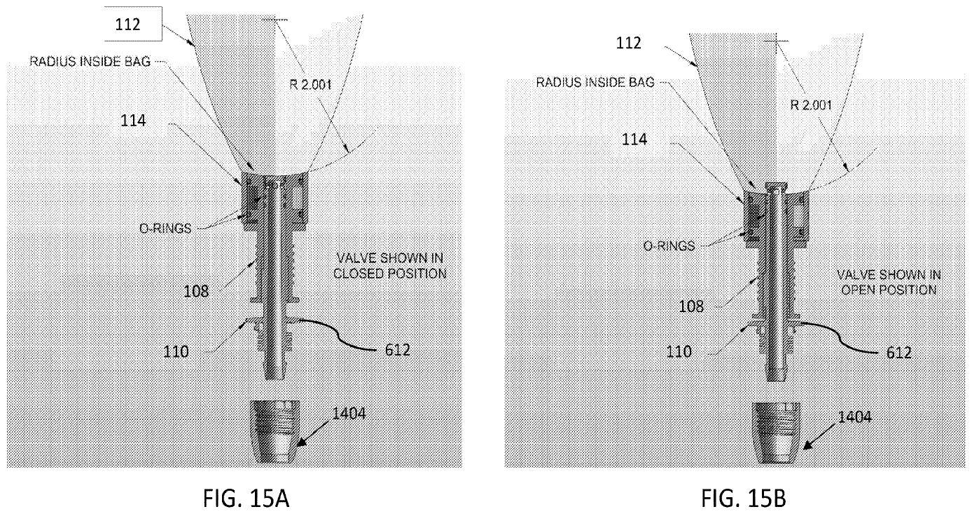

FIG. 15A is a cross-sectional end view of the canoe assembly 402 of FIG. 14A in its “closed” configuration, and FIG. 15B is a cross-sectional end view of the canoe assembly 402 of FIG. 14D in its “open” configuration.

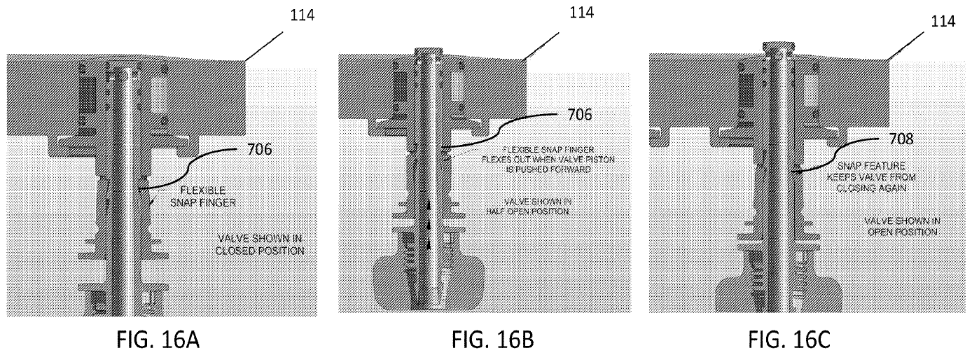

FIG. 16A is a zoomed-in, cross-sectional side view of the canoe assembly 402 of FIG. 14A with the port sizer locking clips 606 engaged with the valve connector “closed” locking tabs 706. FIG. 16C is a zoomed-in, cross-sectional side view of the canoe assembly 402 of FIG. 14D with the port sizer locking clips 606 engaged with the valve connector “open” locking tabs 708. FIG. 16B is a zoomed-in, cross-sectional side view of the same canoe assembly 402 transitioning from the “closed” configuration of FIG. 14A to the “open” configuration of FIG. 14D, during which, the port sizer locking clips 606 flex outward due to the beveled outer diameter of the valve connector “closed” locking tabs 706. Note that the tubes are not shown in FIGS. 16A-16C.

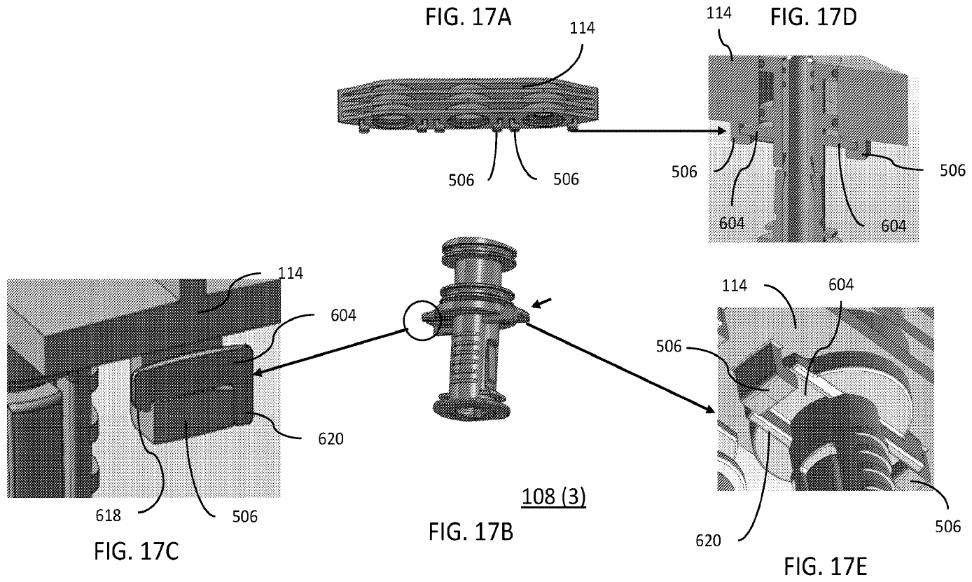

FIGS. 17A and 17B show perspective views of the canoe 114 and the port sizer 108(3), respectively. FIGS. 17C and 17D show different, zoomed-in, cutaway, perspective views, and FIG. 17E shows a zoomed-in, perspective view showing how the port sizer locking arms 604 with their rotation stops 620 and detents 618 engage with the canoe retention clips 506.

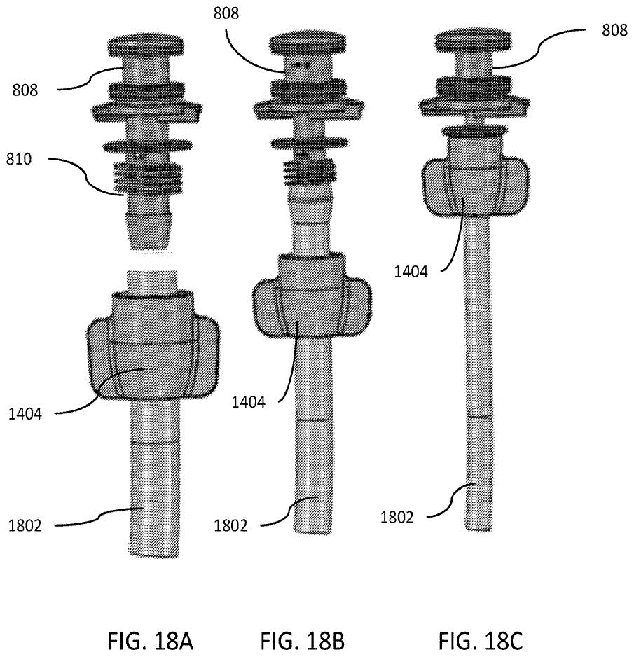

FIG. 18A shows a side view of a large tube (aka tubing) 1802 having a large tube retainer 1404 prior to a large tube 1802 being attached to the threaded tube barb 810 of a large, threaded, fixed port fitting 808. FIG. 18C shows a side view of a medium tube 1802 having a medium tube retainer 1404 after a medium tube 1802 has been attached to the threaded tube barb 810 of a medium, threaded, fixed port fitting 808, but before the tube retainer 1404 is connected to the threaded tube barb 810. FIG. 18C shows a side view of a small tube 1802 having a small tube retainer 1404 after a small tube 1802 has been attached to the threaded tube barb 810 of a small threaded fixed port fitting 808 and after the tube retainer 1404 has been connected to the threaded tube barb 810.

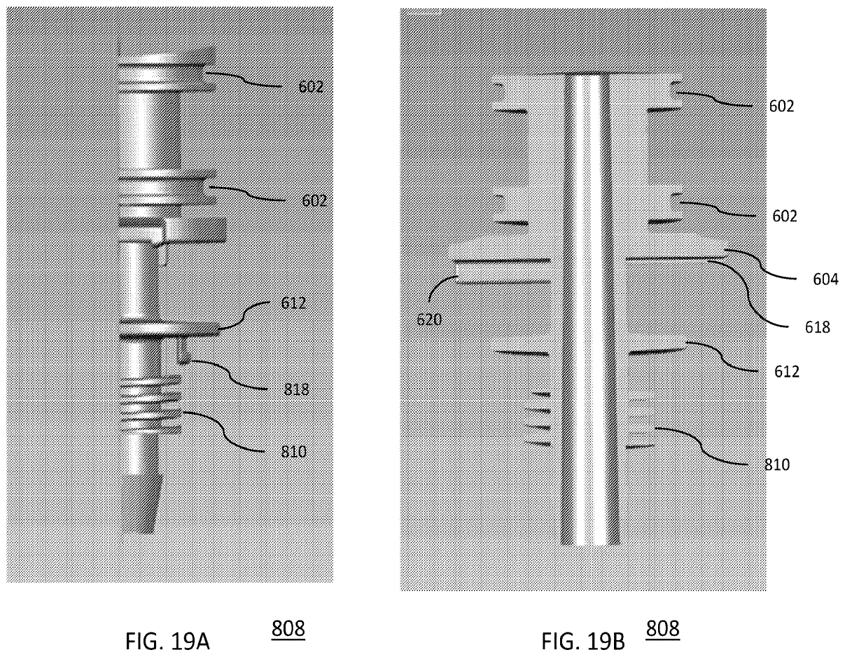

FIG. 19A is a side view of half of a threaded fixed port fitting 808, and FIG. 19B is a cross-sectional side view of the threaded fixed port fitting 808 of FIG. 19A.

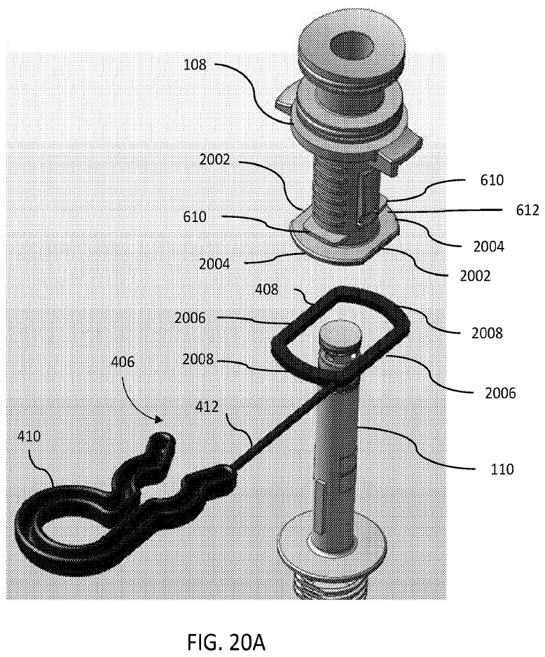

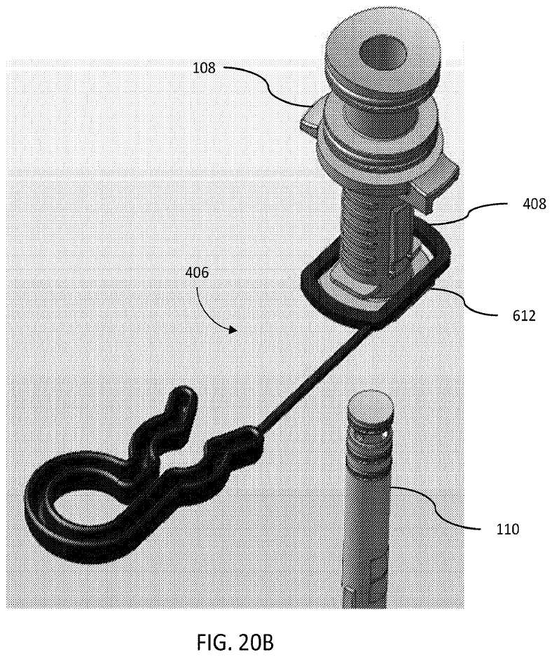

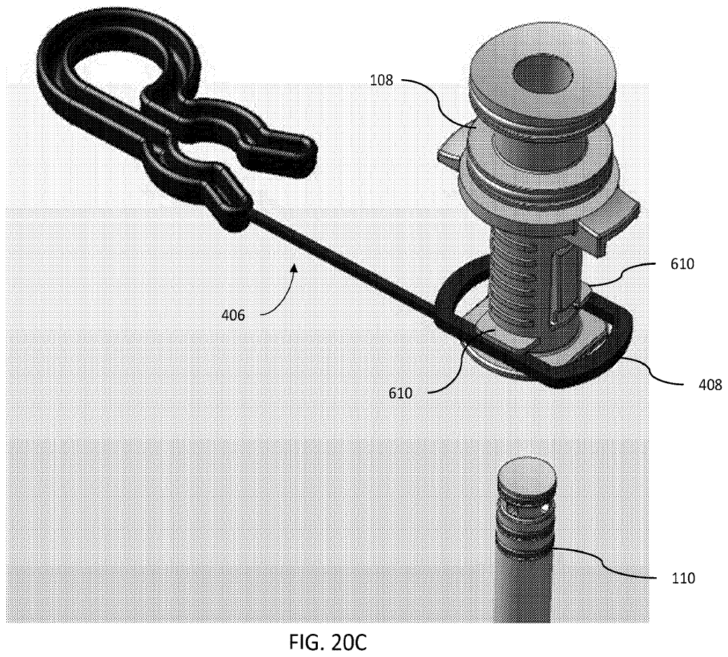

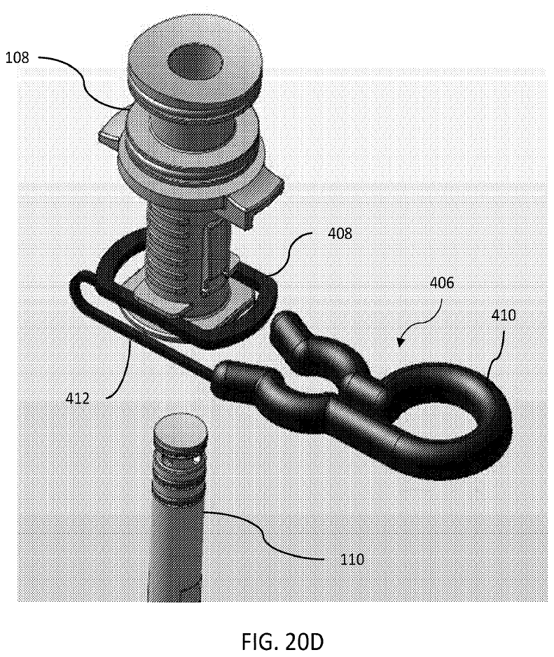

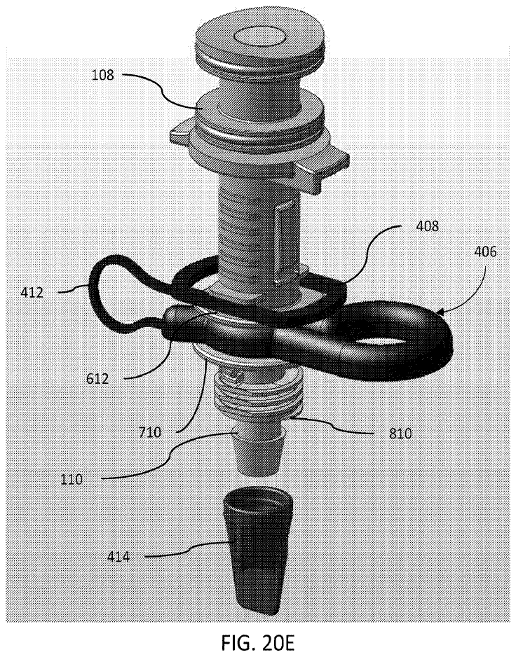

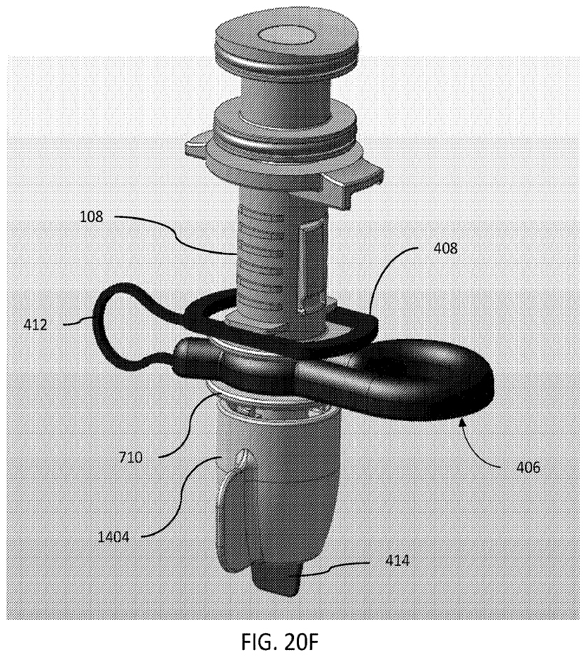

FIG. 20A shows a perspective view of a port sizer 108, a valve connector 110, and a tethered safety clip 406 before they are assembled together. As shown in FIG. 20A, the port sizer 108 has (i) a stop 612 having two opposing, straight edges 2002 and two opposing, curved edges 2004 and (ii) two tether tabs 610. In addition, the tethered safety clip 406 has a tether ring 408 and a tether clip 410 interconnected by a flexible tether 412, where the tether ring 408 has two opposing, straight portions 2006 and two opposing, curved portions 2008.

FIG. 20B shows a perspective view of the elements of FIG. 20A after the tether ring 408 has been placed over the port sizer stop 612. Note that the width of the tether ring 408 is slightly smaller than the width of the port sizer stop 612, but that the length of the tether ring 408 is slightly larger than the length of the port sizer stop 612, thereby enabling the port sizer stop 612 to be worked through the tether ring 408.

FIG. 20C shows a perspective view of the elements of FIG. 20B after the tether ring 408 has been rotated clockwise about 90 degrees with respect to the port sizer 108. Note that the thickness of the tether ring 408 is slightly greater than the distance between the top surface of the port sizer stop 612 and the bottom surfaces of the tether tabs 610 such that the tether ring 408 is held in place by the compressive force applied by the port sizer stop 612 and the tether tabs 610.

FIG. 20D shows a perspective view of the elements of FIG. 20C after the tether clip 410 has been rotated 180 degrees with respect to the tether ring 408, resulting in a bending of the tether 412.

FIG. 20E shows a perspective view of the elements of FIG. 20D and a dust cap 414 after (i) the valve connector 110 has been inserting into the port sizer 108 up to the “closed” configuration and (ii) the tether clip 410 has been pushed onto the valve connector 110 just below the port sizer stop 612 and just above the valve connector stop 710, thereby preventing the valve connector 110 from inadvertently being transitioned from the “closed” configuration to the “open” configuration. FIG. 20E shows the threaded tube barb 810 of the valve connector 110.

FIG. 20F shows a perspective view of the elements of FIG. 20E and a tube retainer 1404 after (i) the dust cap 414 has been placed over the barbed end of the threaded tube barb 810 (not visible in FIG. 20F) and (ii) a tube retainer 1404 has been screwed onto the threaded tube barb 810. The dust cap 414 is in place during pre-assembly, storage, and transportation. When the final assembly is completed, the dust cap will be removed and discarded.

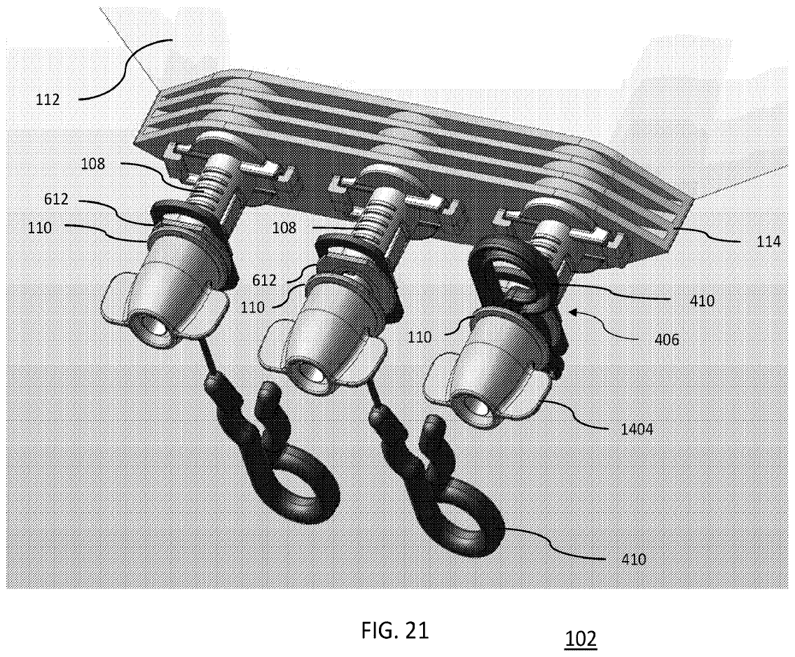

FIG. 21 shows a perspective view of the bottom of a canoe assembly 402 having three port sizer/valve connector sub-assemblies in three different configurations. In the right-most sub-assembly, the valve connector 110 is in the “closed” configuration and the tether clip 410 is engaged on the valve connector 110, as in FIG. 20F except without the dust cap 414. In the middle sub-assembly, the tether clip 410 has been disengaged from the valve connector 110 with the valve connector 110 still in the “closed” configuration. In the left-most sub-assembly, the valve connector 110 has been moved to the “open” configuration with the valve connector stop 710 abutting the port sizer stop 612. Note that the tubes that would be connected to the valve connectors 110 and covered by the tube retainers 1404 are not shown in this view.

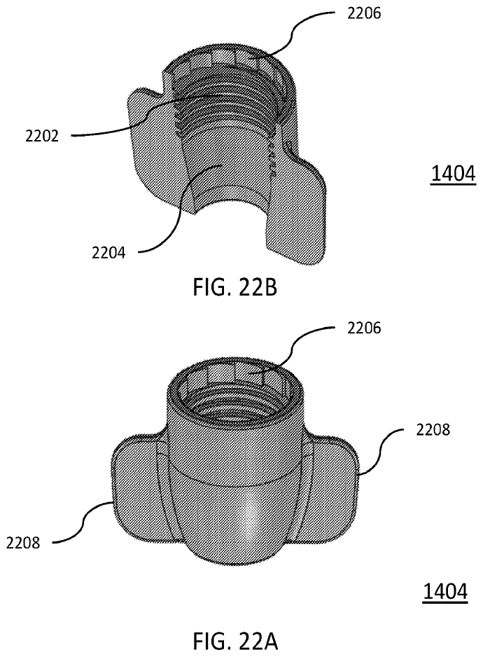

FIG. 22A is a perspective view of a tube retainer 1404. FIG. 22B is a cross-sectional perspective view of the tube retainer 1404 of FIG. 22A. As shown in FIGS. 22A and 22B, the tube retainer 1404 has a threaded cylindrical portion 2202 that engages with a threaded tube barb 810 and a smooth frustum portion 2204 that squeezes an intervening tube between the tube retainer 1404 and the barbed end of a threaded tube barb 810 to prevent leakage at the tube/tube barb interface. The tube retainer 1404 also has anti-rotation features 2206 configured to engage the retainer locking tabs 818 on the valve connector stop 710 of a threaded tube barb 810, thereby preventing the tube retainer 1404 from being removed from a threaded tube barb 810 once it has been engaged. The tube retainer 1404 has two opposing wings 2208 that provide leverage for rotating the tube retainer 1404 onto the threaded tube barb 810.

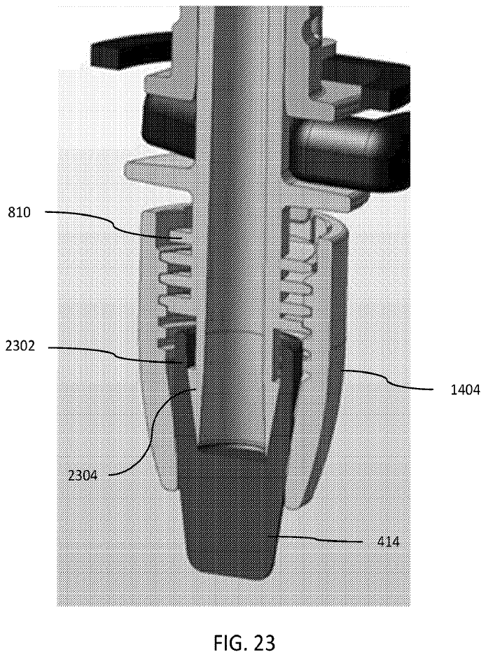

FIG. 23 is a zoomed-in cross-sectional perspective view of a tube retainer 1404 screwed onto a threaded tube barb 810 with an intervening dust cap 414. The dust cap 414 has a small detent feature 2302 that engages with the barbed end 2304 of the tube barb 810 to hold the dust cap 414 onto the tube barb 810, but allows the dust cap 414 to be removed from the tube barb 810. The dust cap 414 prevents the tube retainer 1404 from being fully screwed onto the threaded tube barb 810 and stops the tube retainer 1404 before the anti-rotation features 2206 on the tube retainer 1404 start to engage with the retainer locking tabs 818 of the threaded tube barb 810.

When a tube (not shown in FIG. 23) is to be connected to the threaded tube barb 810, the tube retainer 1404 is removed from the threaded tube barb to enable the dust cap 414 to be removed. At this point, the tube may be inserted through the bottom end of the tube retainer 1404 and onto the barbed end 2304 of the threaded tube barb 810. The tube retainer 1404 may then be fully screwed onto the threaded tube barb 810 such that the anti-rotation features 2206 on the tube retainer 1404 engage with the retainer locking tabs 818 of the threaded tube barb 810 to permanently secure and seal the tube onto the threaded tube barb 810. The design of the tube retainer 1404 accommodates different flexible tube wall thicknesses, effectively compressing the tubing to create a leak-proof seal.

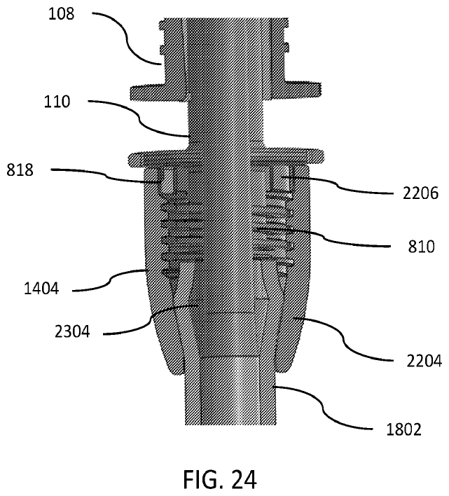

FIG. 24 is a cross-sectional side view of a tube retainer 1404 fully screwed onto a threaded tube barb 810 with a tube 1802 secured and sealed in place and with the anti-rotation features 2206 on the tube retainer 1404 engaged with a retainer locking tab 818 of the threaded tube barb 810. In this position, the smooth frustum portion 2204 of the threaded tube retainer 1404 compresses the tube 1802 against the barbed end 2304 of the valve connector 110 making the seal and keeping the connection secure around the circumference of the connection.

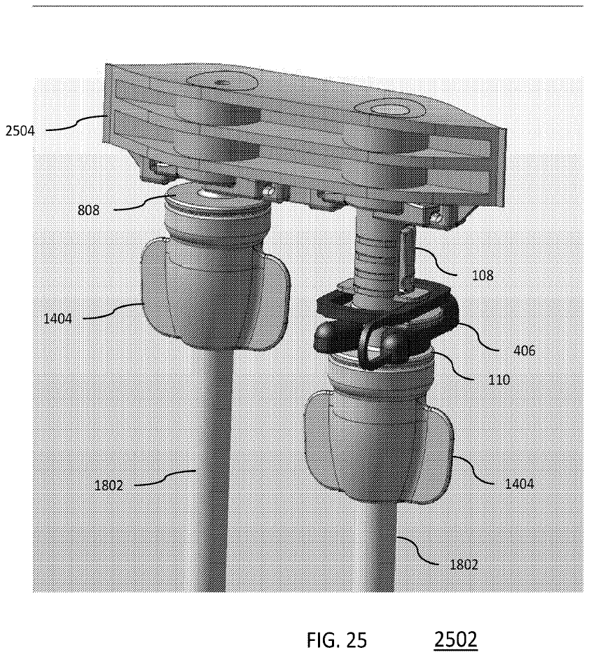

FIG. 25 is a perspective view of a canoe assembly 2502 having a two-port canoe 2504. The sub-assembly on the left has a threaded, fixed port fitting 808 mounted into the canoe 2504 and configured with a tube 1802 secured in place by a tube retainer 1404. The sub-assembly on the right has a port sizer 108 mounted into the canoe 2504 and configured with a valve connector 110 secured in the “closed” configuration by a tethered safety clip 406 and configured with a tube 1802 secured in place by a tube retainer 1404.

In certain embodiments, the bag chamber 112 may be made of multiple layers of HDPE/EVOH/Nylon or COC-PE/Cyclic Olefin for non-binding. The various other plastic elements may be made of polyethylene (PE), polypropylene (PP), polyvinylidene difluoride (PVDF), or other barrier and breathable layers.

### Alternative Ports and Port Fittings

Although port fittings have been described in the context of so-called two-dimensional (2D) bags having canoes, such as bag assembly 100 of FIG. 1, in alternative embodiments, port fittings of the disclosure can be used in the context of other types of containers, such as (without limitation) so-called three-dimensional (3D) bags, bottles, and carboys.

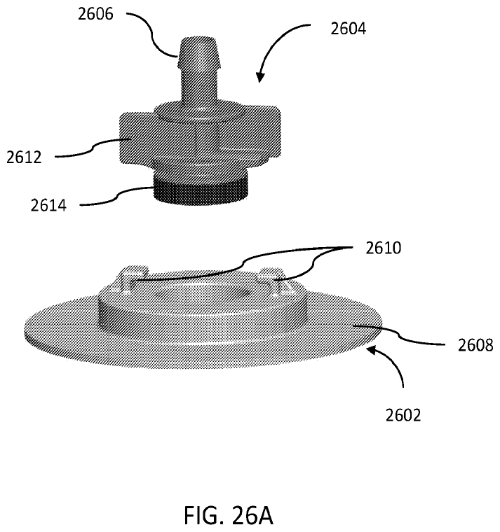

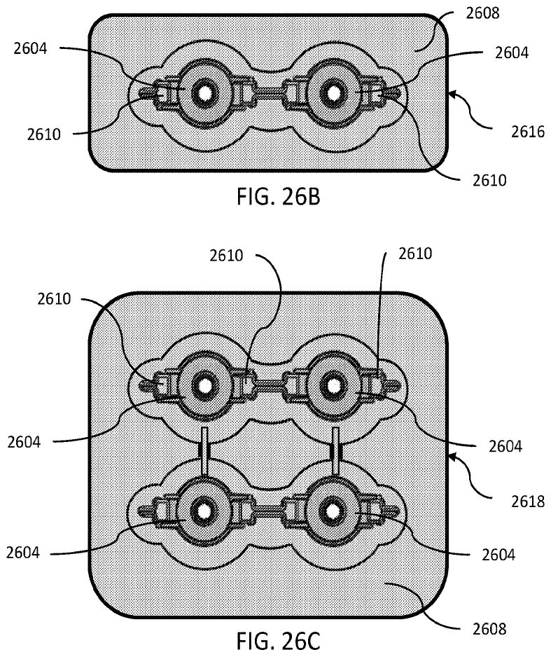

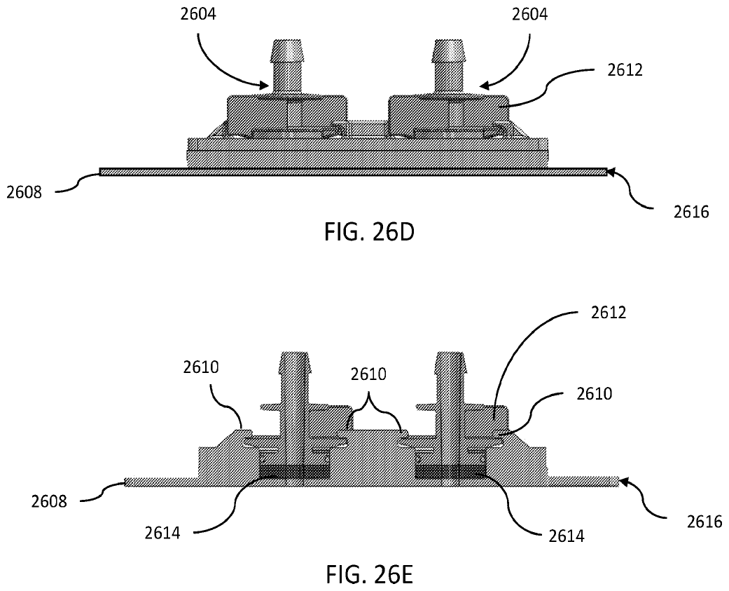

FIG. 26A is an exploded perspective view of a single-fitting port 2602 configured to receive a single, fixed port fitting 2604 having a tube barb 2606. The port 2602 has (i) a port welding flange 2608 at which the port 2602 is thermally welded, e.g., to a 3D bag or bottle/carboy cap (not shown) and (ii) retention clips 2610 that are analogous to the retention clips 506 of FIG. 5. The port 2602 may be made of any suitable material, such as (without limitation) thermoplastic elastomers, thermoplastic vulcanites, thermosets, and fluoropolymers. The port fitting 2604 has two opposing locking arms 2612 that engage the retention clips 506 to lock the port fitting 2604 onto the port 2602. The port fitting 2604 receives a gasket 2614 that forms a seal between the port fitting 2604 and the port 2602.

FIG. 26B is a top-down view of a two-fitting port 2616 configured to receive two instances of the port fitting 2604, and FIG. 26C is a top-down view of a four-fitting port 2618 configured to receive four instances of the port fitting 2604.

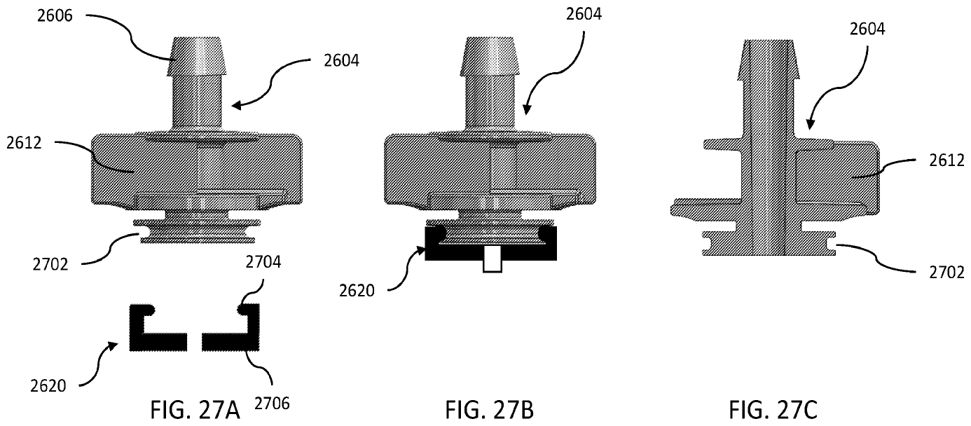

FIGS. 26D and 26E are side and cross-sectional (through the center line) views, respectively, of the two-fitting port 2616 with port fittings 2604 of FIG. 26B and gaskets 2614. FIGS. 27A and 27B are exploded and assembled views, respectively, showing a side view of the fixed port fitting 2604 and a cross-sectional view of a combined gasket/O-ring 2620. FIG. 27C is a cross-sectional side view of the fixed port fitting 2604.

As shown in FIGS. 27A-27C, port fitting 2604 has an O-ring groove 2702, and gasket/O-ring 2620 has an O-ring portion 2704 that is received by the O-ring groove 2702 and a gasket portion 2706 that abuts the bottom of the port fitting 2604. Note that gasket 2614 of FIG. 26E and gasket/O-ring 2620 of FIGS. 27A and 27B provide the same function of forming seals to prevent leakage between the port fitting 2604 and the corresponding port X090.

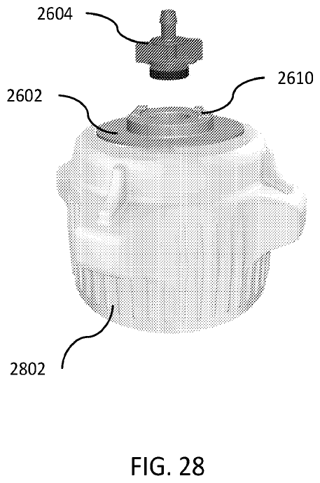

FIG. 28 is a partially exploded view of the port fitting 2604 and the single-fitting port 2602 mated with a cap 2802 for a bottle or carboy (not shown). According to one possible assembly technique, the single-fitting port 2602 is inserted into the bottom of the cap 2802 with the screwing down of the cap 2802 onto the bottle/carboy helping to form the seal between the single-fitting port 2602 and the cap 2802. Another possible assembly technique is to weld the single-fitting port 2602 to the cap 2802. Note that, in general, caps may be configured with single- or multi-fitting ports X090.

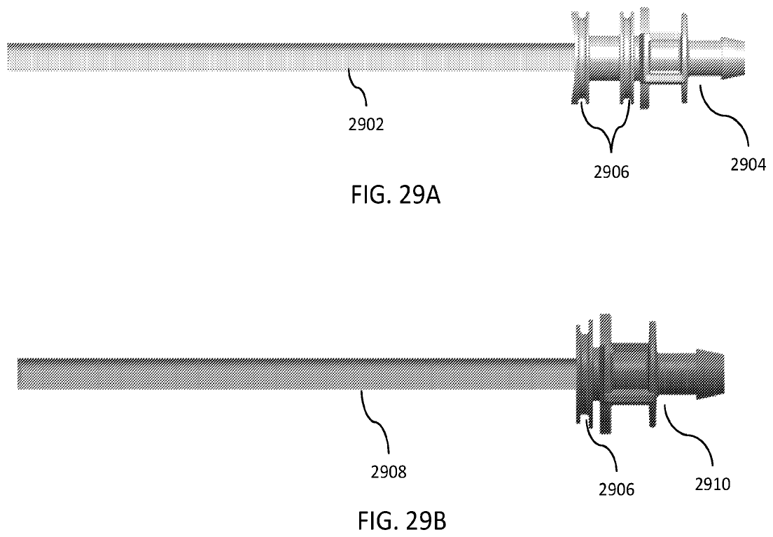

FIG. 29A is a side view of a dip tube 2902 having a dip tube fitting 2904 with two O-ring grooves 2906, and FIG. 29B is a side view of a dip tube 2908 having a dip tube fitting 2910 with single O-ring groove 2906. The fittings 2904 and 2910 enable the dip tubes 2902 and 2908 to be mounted onto container having suitable ports of the present disclosure.

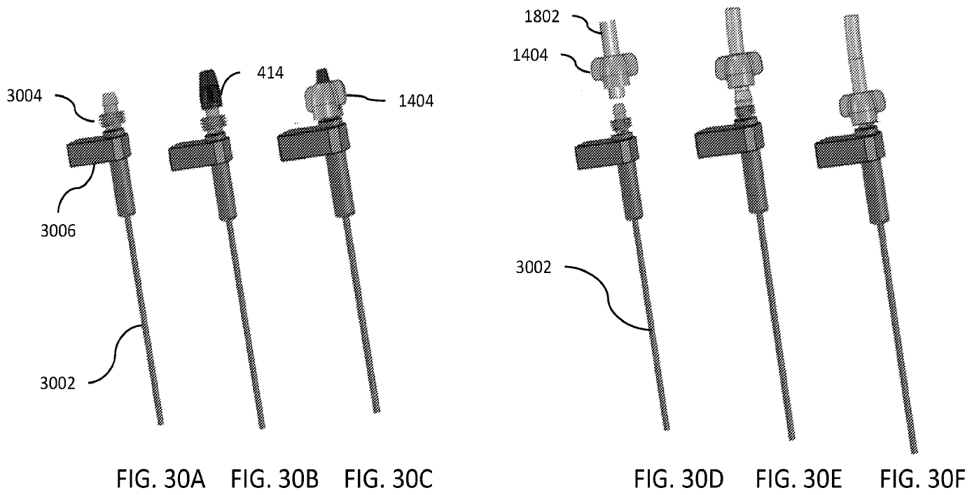

FIG. 30A is a perspective view of a filling needle 3002 having a threaded connector 3004 similar to the threaded tube barb 810 and a mounting bar 3006. FIG. 30B is a perspective view of the filling needle 3002 with a dust cap 414. FIG. 30C is a perspective view of the filling needle 3002 with both the dust cap 414 and a threaded tube retainer 1404. FIGS. 30D-30F are perspective views representing a tube 1802 being mounted onto the filling needle 3002 using the threaded tube retainer 1404.

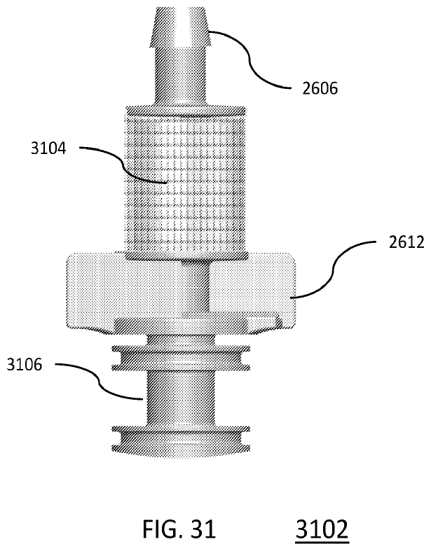

FIG. 31 is a side view of a vented port fitting 3102 having a filter housing 3104 that enables either one-way or two-way air flow into or out of the port fitting channel. The port fitting base 3106 is similar to analogous to the structure of the other port fittings of the disclosure. In one embodiment, the filter housing 3104 allows one-way air flow out of the port fitting channel, thereby enabling air to be vented from the container to which the port fitting 3102 is mounted as the container is being filled with liquid.

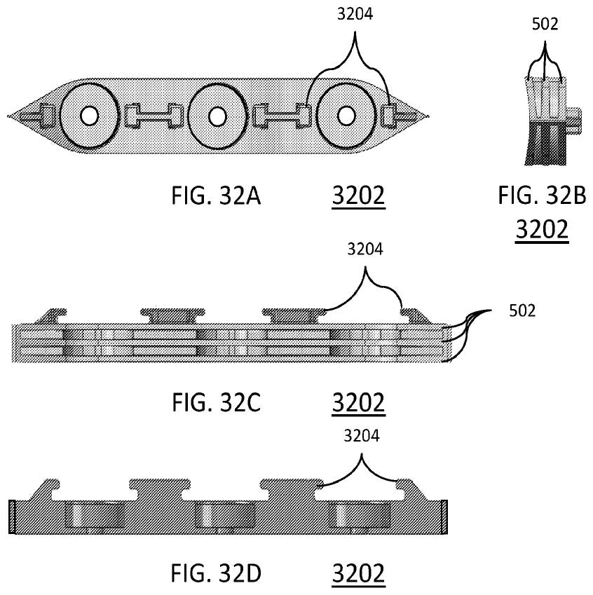

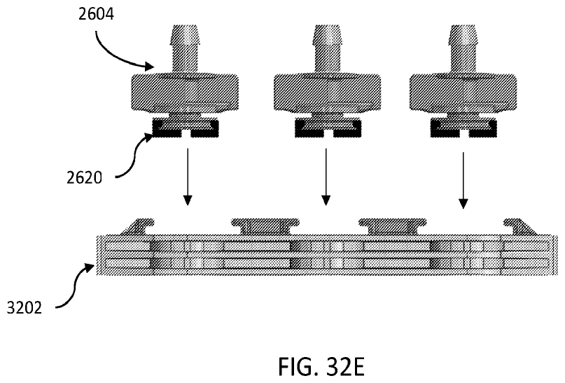

FIGS. 32A, 32B, 32C, and 32D show top-down, end, X-ray side, and cross-sectional side views of a three-port canoe 3202 according to an alternative embodiment. FIG. 32E is an exploded side view of the three-port canoe 3202 ready to receive three fixed port fittings 2604 having gasket/O-rings 2620 to form seals between the canoe 3202 and the port fittings 2604. With a reduced profile compared to canoe 114, canoe 3202 has only three weld bars 502. Note that the retention clips 3204 have a sturdier design than the retention clips 506 of the canoe 114.

The tube retainer system of the present disclosure, which include a threaded tube barb 810 and a threaded tube retainer 1404, can also be applied in fixed tube-to-tube connectors, such as connector 3302 and reducer 3304 shown in the side view of FIGS. 33A and 33B, respectively. FIG. 33C is a side view of a two-piece tube-to-tube connector 3306 having a port sizer analog 3308 and a valve connector analog 3310 that provide flow-valve functionality. FIGS. 33D-33F show side views of retainer-ready cross, tee, and wye tube-to-tube connectors, respectively. FIGS. 33G and 33H show side views of retainer-ready tri-clamp tube barb connectors. FIGS. 331 and 33J show side views of two different three-piece tube-to-tube TEE connectors, each having a tube-to-tube connector analog 3312, a port sizer analog 3308, and a valve connector analog 3310 that provide flow-valve functionality at their lower ports with a threaded tube barb and a luer lock, respectively.

FIG. 33K is a side view of the three-port tube-to-tube connector analog 3312 of FIGS. 331 and 33J with a probe 3314 connected to the lower port to enable fluid path measurements.

FIG. 34 is a side view of a fixed port fitting 3402 having a threaded tube barb with one or more (in this case, three) tube grippers 3404 that further improve the grip of the port fitting 3402 to flexible tubing (not shown).

FIG. 35 is an exploded side view of a reconfigurable assembly 3502 comprising valve connector 3504, port sizer 3506, and tethered safety clip 406. Valve connector 3504 is similar to valve connector 110 except that valve connector 3504 has exterior threading 3508, and port sizer 3506 is similar to port sizer 108 except that port sizer 3506 has interior threading (not shown) that is designed to engage the threading 3508 of the valve connector 3504.

In this embodiment, the valve connector 3504 can be inserted into the port sizer 3506 and then rotated clockwise to a closed position in which the assembly 3502 is configured in a closed configuration that prevents fluid from flowing through the assembly 3502. The valve connector 3504 can then be further rotated clockwise to an open position in which the assembly 3502 is configured in an open configuration that enables fluid to flow through the assembly 3502. The valve connector 3504 can then be rotated counterclockwise to return the assembly 3502 to the closed configuration. In addition, valve connector 3504 can be further rotated counterclockwise to completely remove the valve connector 3504 from the port sizer 3506. When assembled, the tethered safety clip 406 can be used in a manner analogous to that described previously to prevent the valve connector 3504 from inadvertently advancing from the closed position to the open position.

Note that, in order to enable the valve connector 3504 to rotate inside the port sizer 3506, certain features, such as the locking tabs 706/708 and the keyway guide 718 of the valve connector 110 and the keyway 616 and the locking clip 606 of the port sizer 108, may need to be modified or even removed in the designs of the valve connector 3504 and the port sizer 3506.

Although the disclosure has been described in the context of port sizers (e.g., 108) that receive valve connectors (e.g., 110) having locking mechanisms (e.g., 606, 706, 708) that (i) prevent the valve connector from being removed from the port sizer after the valve connector has reached the closed position and (ii) prevent the valve connector from moving back to the closed position after the valve connector has reached the open position, the disclosure also covers embodiments in which (a) the valve connector can be moved back to the closed position from the open position and/or (b) the valve connect can be removed from the port sizer from the closed position. One possible implementation would involve corresponding threads on the outer diameter of the valve connector and the inner diameter of the port sizer that would allow selective clockwise or counterclockwise rotation of the valve connector with respect to the port sizer to move between closed and open positions. Such an implementation would involve modification and possibly removal of some of the existing features of the port sizer 108 and the valve connector 110, such as the locking mechanisms and keying features.

Although the disclosure has been described in the context of containers (e.g., 102) that receive interchangeable port sizers/fittings (e.g., 108/808), where the port fittings (e.g., 808) or valve connectors (e.g., 110) that are inserted into the port sizers (e.g., 108) have threaded tube barbs (e.g., 810) that receive threaded tube retainers (e.g., 1404), the disclosure also includes containers having permanent ports, where the permanent ports or analogous valve connectors that are inserted into the permanent ports have analogous threaded tube barbs that receive analogous threaded tube retainers.

Bag assemblies of the present disclosure may provide one or more of the following features:
Configurable with different port sizes as needed;
Fixed or flow controlling ports;
Standard fitment materials that are being used today;
Low extractable film-non protein binding;
Retainer system for tubing requiring no tools;
Modular construction;
With or without hanger option-snaps on;
One or more ports;
Can be port-size configured in minutes;
Flow lock or fixed ports;
Optional tube retention fittings-no tools required;
Canoe-shaped bag fitment with multiple (e.g., four) sealing bars for increased material welding/sealing surface area, which significantly reduces potential for leaks at weld points;
Concave shape on canoe, port sizer/fitting, and valve connector chamber-facing surfaces for efficient draining and no or low fluid retain;
Dust covers to reduce/eliminate possibility of contaminants entering chamber prior to final assembly (as well as storage and transport);
Filter-ready ports;
Modular designed ports can be configured with multiple sizes and multiple type industry connections and shapes on ends with no tools required to install;
Two-position valves having open and closed positions with flow stop ports-block flow until functioned;
Remains in lock closed position until deployed;
Retainer ready ports or standard ports;
Angle seal on port/retainer;
Modular hanger that can be added at any time;
Anti-slip features for ease of assembly with gloves;
Connector design provides for leak-proof tubing connections;
Variety of probe and sensors allowed to interface via the port sizer/fitting and canoe; and
Fluid Sampling system with or without dosing chambers.

### Figure Labels

| | |
|---|---|
| 102 | bag assembly |
| 104 | bag |
| 106 | bag hangar |
| 108 | port sizer |
| 110 | valve connector |
| 112 | bag chamber |
| 114 | three-port canoe |
| 402 | three-port canoe assembly |
| 404 | curved top surface of canoe 114 |
| 406 | tethered safety clip |
| 408 | tether ring |
| 410 | tether clip |
| 412 | tether |
| 414 | dust cap |
| 502 | weld bar |
| 504 | canoe through-hole |
| 506 | canoe retention clip |
| 508 | canoe tapered end |
| 602 | port sizer O-ring groove |
| 604 | port sizer locking arm |
| 606 | port sizer locking clip |
| 608 | port sizer anti-slip grip |
| 610 | tether tab |
| 612 | port sizer stop |
| 614 | port sizer channel |
| 616 | port sizer keyway |
| 618 | locking arm detent |
| 620 | port sizer rotation stop |
| 622 | locking clip connection |
| 624 | locking clip lower portion |
| 626 | locking clip upper portion |
| 628 | curved port sizer top surface |
| 702A | upper valve connector O-ring groove |
| 702B | lower valve connector O-ring groove |
| 704 | valve connector side port |
| 706 | valve connector “closed” locking tab |
| 708 | valve connector “opened” locking tab |
| 710 | valve connector stop |
| 712 | valve connector tube barb |
| 714 | valve connector channel |
| 716 | curved valve connector top surface |
| 718 | valve connector keyway guide |
| 802 | unthreaded, fixed port fitting |
| 802A | unthreaded, fixed port fitting |
| 803 | extended locking arm |
| 804 | fixed port fitting channel |
| 806 | port fitting tube barb |
| 808 | threaded, fixed port fitting |
| 810 | threaded tube barb |
| 812 | port fitting threading |
| 814 | fixed port fitting having a threaded “wye” tube connector |
| 816 | fixed port fitting having a threaded “tee” tube connector |
| 818 | retainer locking tab |
| 902 | luer-lock valve connector |
| 904 | luer lock |
| 1002 | block-out plug |
| 1004 | curved plug top surface |
| 1006 | plug O-ring groove |
| 1008 | plug locking arm |
| 1102 | “tee” valve connector |
| 1104 | “wye” valve connector |
| 1106 | “wye” valve connector |
| 1108 | valve connector housing |
| 1202 | female bag hangar component |
| 1204 | male bag hangar component |
| 1206 | bag hanger male element |
| 1208 | bag chamber opening |
| 1210 | bag hander female element |
| 1302 | bag probe |
| 1402 | valve connector threading |
| 1404 | tube retainer |
| 1802 | tube |
| 2002 | straight edge of non-circular port sizer stop 612 |
| 2004 | curved edge of non-circular port sizer stop 612 |
| 2006 | straight portion of tether ring 408 |
| 2008 | curved portion of tether ring 408 |
| 2202 | threaded cylindrical portion of tube retainer 1404 |
| 2204 | smooth frustum portion of the tube retainer 1404 |
| 2206 | anti-rotation features of the tube retainer 1404 |
| 2208 | tube retainer wing |
| 2302 | dust cap detent feature |
| 2304 | barbed end of threaded tube barb 810 |
| 2502 | two-port canoe assembly |
| 2504 | two-port canoe |
| 2602 | single-fitting port |
| 2604 | fixed port fitting |
| 2606 | tube barb |
| 2608 | port welding flange |
| 2610 | retention clips |
| 2612 | locking arm |
| 2614 | gasket |
| 2616 | two-fitting port |
| 2618 | four-fitting port |
| 2620 | gasket/O-ring |
| 2702 | gasket groove |
| 2704 | O-ring portion |
| 2706 | gasket portion |
| 2802 | cap |
| 2902 | dip tube |
| 2904 | dip tube fitting |
| 2906 | O-ring groove |
| 2908 | dip tube |
| 2910 | dip tube fitting |
| 3002 | filling needle |
| 3004 | retainer-ready threaded connector |
| 3006 | filling needle mounting bar |
| 3102 | vented port fitting |
| 3104 | filter housing |
| 3106 | port fitting base |
| 3202 | canoe |
| 3204 | retention clips |
| 3302 | fixed tube-to-tube connector |
| 3304 | fixed tube-to-tube connector |
| 3306 | tube-to-tube connector with flow valve |
| 3308 | port sizer analog |
| 3310 | valve connector analog |
| 3312 | tube-to-tube connector analog |
| 3314 | probe |
| 3402 | fixed port fitting |
| 3404 | tube gripper |
| 3502 | reconfigurable valve connector/port sizer assembly |
| 3504 | valve connector |
| 3506 | port sizer |
| 3508 | valve connector threading |

In certain embodiments of the present disclosure, an assembly comprises a port fitting (e.g., 108/802/808/814/816) for a container (e.g., 104). The port fitting comprises (i) at least one O-ring groove (e.g., 602) at a first end of the port fitting and configured to receive at least one O-ring and (ii) two opposing locking arms (e.g., 604) between the at least one O-ring groove and the second end of the port fitting. The port fitting is configured to be mounted onto the container by (a) inserting the first end of the port fitting into a port (e.g., 504) in the container such that the at least one O-ring forms a seal between the port fitting and the container and (b) rotating the port fitting to engage the two opposing locking arms with two opposing retention clips (e.g., 506) of the container.

In at least some of the above embodiments, each locking arm comprises (1) a rotation stop (e.g., 620) configured to stop the rotation of the port fitting relative to the container at one of the retention clips and (2) a detent (e.g., 618) configured to inhibit removal of the port fitting from the container by rotation.

In at least some of the above embodiments, the first end of the port fitting has a curvilinear shape (e.g., 628) configured to match a curvilinear shape of an interior of the container at the port fitting when the port fitting is mounted onto the container.

In at least some of the above embodiments, the assembly further comprises the container having one or more ports (e.g., 504) and two opposing retention clips (e.g., 506) for each port, each port configured to receive any one of two or more port fittings having differently sized passageways enabling different flow rates.

In at least some of the above embodiments, the container comprises two or more of the ports.

In at least some of the above embodiments, the container is a flexible bag chamber (e.g., 112) permanently connected to a rigid canoe (e.g., 114) having the one or more ports (e.g., 504) and the two opposing retention clips (e.g., 506) for each port.

In at least some of the above embodiments, the canoe has a curvilinear inner surface (e.g., 404) configured to match a curvilinear shape of the interior of the flexible bag chamber at the canoe.

In at least some of the above embodiments, the port fitting is a fixed port fitting (e.g., 802/808/814/816) having (i) a channel (e.g., 614/804) from an open first end of the port fitting to an opposing open second end of the port fitting and (ii) a tube barb (e.g., 806) at the open second end of the port fitting and configured to receive a flexible tube (e.g., 1802) over the tube barb.

In at least some of the above embodiments, the port fitting (e.g., 814/816) has one or more additional open ends.

In at least some of the above embodiments, the port fitting (e.g., 808/814/816) further comprises threading (e.g., 812) between the tube barb and the locking arms, wherein the flexible tube is configured to be secured to the tube barb by (a) feeding the flexible tube through an opening in a threaded tube retainer (e.g., 1404); (b) inserting an end of the flexible tube over the tube barb; and (c) rotating the threaded tube retainer onto the port fitting's threading.

In at least some of the above embodiments, the assembly further comprises the threaded tube retainer.

In at least some of the above embodiments, the port fitting further comprises a port fitting stop (e.g., 612) between the threading and the locking arms, wherein the port fitting stop is configured to stop the rotation of the threaded tube retainer.

In at least some of the above embodiments, the port fitting stop comprises at least one retainer locking tab (e.g., 818) configured to engage at least one anti-rotation feature (e.g., 2206) of the threaded tube retainer to lock the threaded tube retainer in place onto the port fitting.

In at least some of the above embodiments, the assembly further comprises a valve connector (e.g., 110/1102/1104/1106/3504) comprising (1) a closed first end; (2) a channel (e.g., 714) from (i) at least one side port (e.g., 704) near the closed first end of the valve connector to (ii) an opposing open second end of the valve connector; (3) at least one upper O-ring groove (e.g., 702A) between (i) the closed first end of the valve connector and (ii) the at least one side port and configured to receive at least one upper O-ring; (4) at least one lower O-ring groove (e.g., 702B) between (i) the at least one side port and (ii) the open second end of the valve connector and configured to receive at least one lower O-ring; and (5) a tube barb (e.g., 712) at the open second end of the valve connector and configured to receive a flexible tube (e.g., 1802) over the tube barb. The valve connector is configured to be mounted onto the port fitting into a closed configuration by inserting the closed first end of the valve connector into the second end of the port fitting to a closed position such that the upper and lower O-rings form seals between the valve connector and the port fitting. The valve connector is configured to be moved from the closed configuration to an open configuration by further inserting the valve connector into the port fitting to an open position in which the at least one side port of the valve connector is open to the interior of the container to connect the interior of the container to the channel through the valve connector with the at least one lower O-ring forming at least one seal between the valve connector and the port fitting.

In at least some of the above embodiments, the valve connector (e.g., 1102/1104/1106) has one or more additional open ends.

In at least some of the above embodiments, the closed first end of the valve connector has a curvilinear shape (e.g., 716) configured to match a curvilinear shape of an interior of the container at the valve connector when (i) the port fitting is mounted onto the container and (ii) the valve connector is inserted into the port fitting up to the closed position.

In at least some of the above embodiments, the port fitting comprises a port fitting stop (e.g., 612) at the second end of the port fitting, and the valve connector comprises a valve connector stop (e.g., 710) between the at least one lower O-ring groove and the tube barb, wherein the valve connector stop and the port fitting stop are configured to stop the insertion of the valve connector into the port fitting at the open position.

In at least some of the above embodiments, the assembly further comprises a tethered safety clip (e.g., 406) configured to be removably positioned between the valve connector stop and the port fitting stop to prevent the valve connector from moving from the closed position to the open position.

In at least some of the above embodiments, the valve connector further comprises threading (e.g., 1402) between the tube barb and the valve connector stop, wherein the flexible tube is configured to be secured to the tube barb by (a) feeding the flexible tube through an opening in a threaded tube retainer (e.g., 1404); (b) inserting an end of the flexible tube over the tube barb; and (c) rotating the threaded tube retainer onto the port fitting's threading, wherein the valve connector stop is configured to stop the rotation of the threaded tube retainer.

In at least some of the above embodiments, the assembly further comprises the threaded tube retainer.

In at least some of the above embodiments, the valve connector stop comprises at least one retainer locking tab (e.g., 818) configured to engage at least one anti-rotation feature (e.g., 2206) of the threaded tube retainer to lock the threaded tube retainer in place onto the valve connector.

In at least some of the above embodiments, the port fitting comprises (i) at least one locking clip (e.g., 606) located between the locking arms and the second end of the port fitting and (ii) at least one keyway (e.g., 618) located between the at least one locking clip and the second end of the port fitting, and the valve connector comprises (i) at least one closed locking tab (e.g., 706) located between the at least one lower O-ring groove and the tube barb; (ii) at least one open locking tab (e.g., 708) located between the at least one closed locking tab and the tube barb; and (iii) at least one keyway guide (e.g., 718) located between the at least one open locking tab and the tube barb. The at least one keyway is configured to receive the at least keyway guide to ensure proper rotational orientation when the valve connector is inserted into the port fitting; the at least one locking clip is configured to receive the at least one closed locking tab at the closed position and inhibit removal of the valve connector from the port fitting; and the at least one locking clip is configured to receive the at least one open locking tab at the open position and inhibit moving the valve connector from the open position to the closed position.

In at least some of the above embodiments, the valve connector (e.g., 3504) is movable from the open position back to the closed portion within the port fitting (e.g., 3506) to reconfigure the assembly from the open configuration back to the closed configuration.

In certain embodiments of the present disclosure, an assembly comprises a connector (e.g., 110/808) and a threaded tube retainer (e.g., 1404). The connector comprises a first end; an open second end; a tube barb (e.g., 712/806) at the open second end and configured to receive a flexible tube (e.g., 1802) over the tube barb; threading (e.g., 812/1402) between the first end and the tube barb; and a connector stop (e.g., 710/612) between the first end and the threading. The flexible tube is configured to be secured to the tube barb by feeding the flexible tube through an opening in the threaded tube retainer; inserting an end of the flexible tube over the tube barb; and rotating the threaded tube retainer onto the connector's threading, wherein the connector stop is configured to stop the rotation of the threaded tube retainer.

In at least some of the above embodiments, the connector stop comprises at least one retainer locking tab (e.g., 818) configured to engage at least one anti-rotation feature (e.g., 2206) of the threaded tube retainer to lock the threaded tube retainer in place onto the valve connector.

In at least some of the above embodiments, the threaded tube retainer comprises an inner smooth frustum portion (e.g., 2204) adjacent to the threaded tube retainer's threading and configured to form seals between (i) the tube barb and an inner surface of the flexible tube and between (ii) an outer surface of the flexible tube and the threaded tube retainer.

In at least some of the above embodiments, the threaded tube retainer comprises to opposing wings (e.g., 2208) providing leverage for rotating the threaded tube retainer onto the connector's threading.

In at least some of the above embodiments, the connector is a valve connector (e.g., 110).

In at least some of the above embodiments, the connector is a fixed port fitting (e.g., 808).

In at least some of the above embodiments, the connector is tube-to-tube connector (e.g., 3302-3306, 3312).

While this disclosure includes references to illustrative embodiments, this specification is not intended to be construed in a limiting sense. Various modifications of the described embodiments, as well as other embodiments within the scope of the disclosure, which are apparent to persons skilled in the art to which the disclosure pertains are deemed to lie within the principle and scope of the disclosure, e.g., as expressed in the following claims.

Unless explicitly stated otherwise, each numerical value and range should be interpreted as being approximate as if the word “about” or “approximately” preceded the value or range.

It will be further understood that various changes in the details, materials, and arrangements of the parts which have been described and illustrated in order to explain the nature of this disclosure may be made by those skilled in the art without departing from the scope of the disclosure, e.g., as expressed in the following claims.

Reference herein to “one embodiment” or “an embodiment” means that a particular feature, structure, or characteristic described in connection with the embodiment can be included in at least one embodiment of the disclosure. The appearances of the phrase “in one embodiment” in various places in the specification are not necessarily all referring to the same embodiment, nor are separate or alternative embodiments necessarily mutually exclusive of other embodiments. The same applies to the term “implementation.”

Unless otherwise specified herein, the use of the ordinal adjectives “first,” “second,” “third,” etc., to refer to an object of a plurality of like objects merely indicates that different instances of such like objects are being referred to, and is not intended to imply that the like objects so referred-to have to be in a corresponding order or sequence, either temporally, spatially, in ranking, or in any other manner.

Also for purposes of this description, the terms “couple,” “coupling,” “coupled,” “connect,” “connecting,” or “connected” refer to any manner known in the art or later developed in which energy is allowed to be transferred between two or more elements, and the interposition of one or more additional elements is contemplated, although not required. Conversely, the terms “directly coupled,” “directly connected,” etc., imply the absence of such additional elements. The same type of distinction applies to the use of terms “attached” and “directly attached,” as applied to a description of a physical structure. For example, a relatively thin layer of adhesive or other suitable binder can be used to implement such “direct attachment” of the two corresponding components in such physical structure.

The described embodiments are to be considered in all respects as only illustrative and not restrictive. In particular, the scope of the disclosure is indicated by the appended claims rather than by the description and figures herein. All changes that come within the meaning and range of equivalency of the claims are to be embraced within their scope.

In this specification including any claims, the term “each” may be used to refer to one or more specified characteristics of a plurality of previously recited elements or steps. When used with the open-ended term “comprising,” the recitation of the term “each” does not exclude additional, unrecited elements or steps. Thus, it will be understood that an apparatus may have additional, unrecited elements and a method may have additional, unrecited steps, where the additional, unrecited elements or steps do not have the one or more specified characteristics.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

All documents mentioned herein are hereby incorporated by reference in their entirety or alternatively to provide the disclosure for which they were specifically relied upon.

The embodiments covered by the claims in this application are limited to embodiments that (1) are enabled by this specification and (2) correspond to statutory subject matter. Non-enabled embodiments and embodiments that correspond to non-statutory subject matter are explicitly disclaimed even if they fall within the scope of the claims.

While preferred embodiments of the disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the technology of the disclosure. It is intended that the following claims define the scope of the invention and that methods and structures within the scope of these claims and their equivalents be covered thereby.

1.-23. (canceled)

24. An assembly comprising:
a connector comprising:
a first end and an open second end;
a tube barb at the open second end and configured to receive a flexible tube over the tube barb;
threading between the first end and the tube barb; and
a connector stop between the first end and the threading; and
a threaded tube retainer, wherein the flexible tube is configured to be secured to the tube barb by:
feeding the flexible tube through an opening in the threaded tube retainer;
inserting an end of the flexible tube over the tube barb; and
rotating the threaded tube retainer onto the threading of the connector, wherein the connector stop is configured to stop the rotation of the threaded tube retainer.

25. The assembly of claim 24, wherein the connector stop comprises at least one retainer locking tab configured to engage at least one anti-rotation feature of the threaded tube retainer to lock the threaded tube retainer in place onto the valve connector.

26. The assembly of claim 24, wherein the threaded tube retainer comprises an inner smooth frustum portion adjacent to the threading of the threaded tube retainer and configured to form seals between (i) the tube barb and an inner surface of the flexible tube and between (ii) an outer surface of the flexible tube and the threaded tube retainer.

27. The assembly of claim 24, wherein the threaded tube retainer comprises two opposing wings providing leverage for rotating the threaded tube retainer onto the connector's threading.

28. The assembly of claim 24, wherein the connector is a valve connector.

29. The assembly of claim 24, wherein the connector is a fixed port fitting.

30. The assembly of claim 24, wherein the connector is a tube-to-tube connector.
